SEQUENCE LISTING (32 sequences)

SEQ ID NO: 1 - RNA, 80 nt, synthetic construct
  misc_feature 1..80: note = Synthetic nucleotide
  source 1..80: mol_type = other RNA
gttttagagctagaaatagcaagttaaaataaggctagtccgttatcaacttgaaaaagtggcaccgagtcggtgctttt

SEQ ID NO: 2 - skipped

SEQ ID NO: 3 - DNA, 20 bp, synthetic construct
  misc_feature 1..20: note = Synthetic nucleotide
  source 1..20: mol_type = other DNA
gattaatgacatacgcattt

SEQ ID NO: 4 - DNA, 20 bp, synthetic construct
  misc_feature 1..20: note = Synthetic nucleotide
  source 1..20: mol_type = other DNA
tggaccacatggctttgctc

SEQ ID NO: 5 - DNA, 20 bp, synthetic construct
  misc_feature 1..20: note = Synthetic nucleotide
  source 1..20: mol_type = other DNA
tgagcaaagccatgtggtcc

SEQ ID NO: 6 - DNA, 20 bp, synthetic construct
  misc_feature 1..20: note = Synthetic nucleotide
  source 1..20: mol_type = other DNA
gtggtcctattatgttgatg

SEQ ID NO: 7 - DNA, 20 bp, synthetic construct
  misc_feature 1..20: note = Synthetic nucleotide
  source 1..20: mol_type = other DNA
catagatgaccaagcttggc

SEQ ID NO: 8 - DNA, 20 bp, synthetic construct
  misc_feature 1..20: note = Synthetic nucleotide
  source 1..20: mol_type = other DNA
catagatgaccaagcttggc

SEQ ID NO: 9 - DNA, 20 bp, synthetic construct
  misc_feature 1..20: note = Synthetic nucleotide
  source 1..20: mol_type = other DNA
agcacctgagcaaagccatg

SEQ ID NO: 10 - DNA, 20 bp, synthetic construct
  misc_feature 1..20: note = Synthetic nucleotide
  source 1..20: mol_type = other DNA
agattaatgacatacgcatt

SEQ ID NO: 11 - RNA, 100 nt, synthetic construct
  misc_feature 1..100: note = Synthetic nucleotide
  source 1..100: mol_type = other RNA
  modified_base 29..40: mod_base = OTHER; note = 2'-O-methylated base
  modified_base 68..100: mod_base = OTHER; note = 2'-O-methylated base
  misc_structure 1..4: note = thiolated phosphate
  misc_feature 97..100: note = thiolated phosphate
gattaatgacatacgcatttgttttagagctagaaatagcaagttaaaataaggctagtccgttatcaacttgaaaaagtggcaccgagtcggtgctttt

SEQ ID NO: 12 - DNA, 20 bp, synthetic construct
  misc_feature 1..20: note = Synthetic nucleotide
  source 1..20: mol_type = other DNA
ggatccctcgtataacaata

SEQ ID NO: 13 - DNA, 20 bp, synthetic construct
  misc_feature 1..20: note = Synthetic nucleotide
  source 1..20: mol_type = other DNA
tggactacatagttgtgtga

SEQ ID NO: 14 - DNA, 20 bp, synthetic construct
  misc_feature 1..20: note = Synthetic nucleotide
  source 1..20: mol_type = other DNA
ttcaatcctcttgtcacctg

SEQ ID NO: 15 - DNA, 20 bp, synthetic construct
  misc_feature 1..20: note = Synthetic nucleotide
  source 1..20: mol_type = other DNA
ctgccagactctctgaaccc

SEQ ID NO: 16 - DNA, 20 bp, synthetic construct
  misc_feature 1..20: note = Synthetic nucleotide
  source 1..20: mol_type = other DNA
aaaacctaccgcaagttgcc

SEQ ID NO: 17 - DNA, 20 bp, synthetic construct
  misc_feature 1..20: note = Synthetic nucleotide
  source 1..20: mol_type = other DNA
gacatctctatgtcggccac

SEQ ID NO: 18 - RNA, 20 nt, synthetic construct
  source 1..20: mol_type = other RNA
gattaatgacatacgcattt

SEQ ID NO: 19 - RNA, 20 nt, synthetic construct
  source 1..20: mol_type = other RNA
tggaccacatggctttgctc

SEQ ID NO: 20 - RNA, 20 nt, synthetic construct
  source 1..20: mol_type = other RNA
tgagcaaagccatgtggtcc

SEQ ID NO: 21 - RNA, 20 nt, synthetic construct
  source 1..20: mol_type = other RNA
gtggtcctattatgttgatg

SEQ ID NO: 22 - RNA, 20 nt, synthetic construct
  source 1..20: mol_type = other RNA
catagatgaccaagcttggc

SEQ ID NO: 23 - RNA, 20 nt, synthetic construct
  source 1..20: mol_type = other RNA
catagatgaccaagcttggc

SEQ ID NO: 24 - RNA, 20 nt, synthetic construct
  source 1..20: mol_type = other RNA
agcacctgagcaaagccatg

SEQ ID NO: 25 - RNA, 20 nt, synthetic construct
  source 1..20: mol_type = other RNA
agattaatgacatacgcatt

SEQ ID NO: 26 - RNA, 20 nt, synthetic construct
  source 1..20: mol_type = other RNA
ggatccctcgtataacaata

SEQ ID NO: 27 - RNA, 20 nt, synthetic construct
  source 1..20: mol_type = other RNA
tggactacatagttgtgtga

SEQ ID NO: 28 - RNA, 20 nt, synthetic construct
  source 1..20: mol_type = other RNA
ttcaatcctcttgtcacctg

SEQ ID NO: 29 - RNA, 20 nt, synthetic construct
  source 1..20: mol_type = other RNA
ctgccagactctctgaaccc

SEQ ID NO: 30 - RNA, 20 nt, synthetic construct
  source 1..20: mol_type = other RNA
aaaacctaccgcaagttgcc

SEQ ID NO: 31 - RNA, 20 nt, synthetic construct
  source 1..20: mol_type = other RNA
gacatctctatgtcggccac

SEQ ID NO: 32 - RNA, 100 nt, synthetic construct; identical to SEQ ID NO: 11
  source 1..100: mol_type = other RNA

(19) United States
(12) Patent Application Publication
(10) Pub. No.: US 2025/0228975 A1
(43) Pub. Date: Jul. 17, 2025

(54) METHODS AND COMPOSITIONS FOR TREATING LIPOPROTEIN-RELATED DISEASES

(71) Applicant: CRISPR THERAPEUTICS AG, Zug (CH)

(72) Inventors: Sanjay D'SOUZA, South Boston, MA (US); Laura SERWER, South Boston, MA (US); Jonathan TERRETT, South Boston, MA (US); John KULMAN, South Boston, MA (US)

(21) Appl. No.: 18/849,438

(22) PCT Filed: Mar. 20, 2023
(86) PCT No.: PCT/IB2023/052720

§ 371 Date: Sep. 20, 2024

Related U.S. Application Data
(60) Provisional application No. 63/322,078, filed on Mar. 21, 2022.
(60) Provisional application No. 63/351,542, filed on Jun. 13, 2022.
(60) Provisional application No. 63/385,093, filed on Nov. 28, 2022.

(51) Int. Cl. A61K 48/00; C12N 9/22; C12N 15/11; C12N 15/90

(52) U.S. Cl. CPC A61K 48/0058; C12N 9/22; C12N 15/11; C12N 15/907; C12N 2310/20

(57) ABSTRACT

The present disclosure relates to methods, compositions and kits for modulating the expression of LPA gene and for treating lipoprotein-related diseases, for example cardiovascular diseases, in a subject by gene editing.

### CROSS-REFERENCE TO RELATED APPLICATIONS

This application claims the benefit under 35 U.S.C. § 119(e) to U.S. Provisional Patent Application No. 63/322,078 filed on Mar. 21, 2022, U.S. Provisional Patent Application No. 63/351,542 filed on Jun. 13, 2022, and U.S. Provisional Patent Application No. 63/385,093 filed on Nov. 28, 2022. The contents of these related applications are incorporated herein by reference in their entirety for all purposes.

### REFERENCE TO SEQUENCE LISTING

The present application is being filed along with a Sequence Listing in electronic format. The Sequence Listing is provided as a file entitled 80EM-341712-WO_SeqList, created Mar. 15, 2023, which is 35.9 kilobytes in size. The information in the electronic format of the Sequence Listing is incorporated herein by reference in its entirety.

### BACKGROUND

### Field

The present disclosure generally relates to the field of molecular biology and biotechnology, including gene editing.

### Description of the Related Art

Lipoprotein(a) (Lp(a)) is an atherogenic lipoprotein consisting of the protein apolipoprotein(a) [apo(a)] covalently bound to the apolipoprotein B-100 (apoB) component of a low-density lipoprotein (LDL) particle. High levels of Lp(a) are associated with increased risk of cardiovascular disease including, for example, calcific aortic valve disease (high level of Lp(a) causes higher incidence and faster progression of the disease), myocardial infarctions (MI), coronary heart disease, atherosclerosis, thrombosis and stroke. There are currently no approved medicines that directly target Lp(a).

The targeting of DNA using the RNA-guided, DNA-targeting principle of CRISPR (Clustered Regularly Interspaced Short Palindromic Repeats)-Cas (CRISPR associated) systems has been widely used. CRISPR-Cas systems can be divided in two classes, with class 1 systems utilizing a complex of multiple Cas proteins (such as type I, III, and IV CRISPR-Cas systems) and class 2 systems utilizing a single Cas protein (such as type II, V, and VI CRISPR-Cas systems). Type II CRISPR-Cas-based systems have been used for genome editing, and require a Cas polypeptide or variant thereof guided by a customizable guide RNA (gRNA) for programmable DNA targeting.

There is a need for a safe and effective gene editing strategy to treat lipoprotein-related disease, such as cardiovascular disease and calcific aortic valve disorder, by reducing Lp(a) levels.

### SUMMARY

Disclosed herein include methods, compositions, and kits for treating lipoprotein-related diseases. Some embodiments provide a method for treating a lipoprotein-related disease in a subject in need thereof, comprising administering to the subject a plurality of nanoparticles complexed with (a) a guide RNA (gRNA) targeting LPA gene (LPA gRNA) or a nucleic acid encoding a LPA gRNA; and (b) a nucleic acid encoding a RNA-guided endonuclease, thereby treating the lipoprotein-related disease in the subject. The subject can be administered with the plurality of nanoparticles two or more times. In some embodiments, each two of the two or more administrations are about two weeks to about four weeks apart. In some embodiments, each two of the two or more administrations are at least three months apart. In some embodiments, the method comprises a single administration of the plurality of nanoparticles to the subject.

In some embodiments, the LPA expression in the plasma of the subject is reduced by at least 20%, by at least 30%, by at least 40%, by at least 50%, by at least 60%, by at least 65%, by at least 70%, by at least 75%, by at least 80%, by at least 85%, or by at least 90% after the administration. In some embodiments, the concentration of LPA protein in the plasma of the subject is reduced by at least 20%, by at least 30%, by at least 40%, by at least 50%, by at least 60%, by at least 65%, by at least 70%, by at least 75%, by at least 80%, by at least 85%, or by at least 90% after the administration. In some embodiments, the reduction is for three weeks, four weeks, five weeks, two months, three months, six month, one year, two years, three years, five years, ten years, fifteen years, or longer after the administration. In some embodiments, the reduction is 65% one month after the administration. In some embodiments, the reduction is 75% three months after the administration. In some embodiments, the reduction is relative to (a) the LPA expression or the concentration of LPA protein in the plasma of the subject prior to being administered with the plurality nanoparticles; (b) the LPA expression or the concentration of LPA protein in one or more untreated subjects; and/or (c) a reference level of LPA expression or the concentration of LPA protein of healthy subjects.

The subject in need can have a Lp(a) level of more than 50 mg/dl, for example 60 mg/dl, 70 mg/dl, 80 mg/dl, 90 mg/dl, 100 mg/dl, 110 mg/dl, 120 mg/dl, 130 mg/dl, 140 mg/dl, 150 mg/dl, 200 mg/dl, 250 mg/dl, 300 mg/dl, or more. In some embodiments, the subject in need has a Lp(a) level of more than 100 mg/dL. In some embodiments, the subject in need has an increased risk of myocardial infarction (MI) independent of established cardiovascular disease (CVD) risk factors, or an increased lifetime risk of atherosclerotic cardiovascular disease (ASCVD).

The method described herein can comprise measuring the blood or serum level of Lp(a) in the subject prior to, during, and/or after the administration. In some embodiments, the method comprises identifying a subject in need of the treatment.

The lipoprotein-related disease can be a metabolic disease, a cardiovascular disease, a lipid metabolism disease, or a combination thereof. In some embodiments, the lipoprotein-related disease is calcific aortic valve disease, myocardial infarctions, coronary heart disease, atherosclerosis, thrombosis, stroke, coronary artery disease, familial hyperlipidemia, myocardial infarction, peripheral arterial disease, calcific aortic valve stenosis, or a combination thereof. In some embodiments, one or more symptoms of the lipoprotein-related disease in the subject is reduced or relieved. In some embodiments, the administering to the subject a plurality of nanoparticles complexed reduces cardiovascular risk, likelihood of mortality related to cardiovascular events, or a combination thereof.

In some embodiments, the nucleic acid encoding a RNA-guided endonuclease is a mRNA of the RNA-guided endonuclease, for example Cas9 endonuclease. Non-limiting examples of the Cas9 endonuclease include S. pyogenes Cas9, S. aureus Cas9, N. meningitides Cas9, S. thermophilus Cas9, S. thermophilus 3 Cas9, T. denticola Cas9, and variants thereof. The gRNA can be a single-guide RNA (sgRNA). In some embodiments, the gRNA targets exon 3 of LPA gene. In some embodiments, the LPA gRNA comprises a spacer sequence of any one of SEQ ID NOs: 18-25. In some embodiments, the LPA gRNA comprises the spacer sequence of SEQ ID NO: 18. In some embodiments, the LPA gRNA is a single guide RNA (sgRNA) comprising the sequence of SEQ ID NO: 32. In some embodiments, the LPA gRNA is a single guide RNA (sgRNA) comprising the sequence of SEQ ID NO: 11.

The LPA gRNA or the nucleic acid encoding the LPA gRNA, and the RNA-guided nuclease can be encapsulated in the plurality of nanoparticles. In some embodiments, the nanoparticles are lipid nanoparticles. The subject can be a primate subject, for example human.

In some embodiments, the method comprises a single administration of the plurality of nanoparticles to the subject. For example, the plurality of nanoparticles can be administered to the subject in the single administration at a dose of, or a dose of about, 0.1 mg/kg, 0.3 mg/kg, 0.6 mg/kg, or 1 mg/kg of total nucleic acids of (a) and (b).

In some embodiments, the plurality of nanoparticles are lipid nanoparticles. In some embodiments, the lipid nanoparticles comprise one or more neutral lipids, charged lipids, ionizable lipids, steroids, and polymers conjugated lipids. In some embodiments, the lipid nanoparticles comprise cholesterol, a polyethylene glycol (PEG) lipid, or both.

The method can further comprises determining (i) a level of one or more of alanine transaminase (ALT), aspartate transaminase (AST), gamma-glutamyl transferase (GGT), bilirubin, alkaline phosphatase (Alk Phos) and albumin; (ii) prothrombin time (PT), and/or (iii) partial thromboplastin time (PTT) in the subject before the administration, after the administration, or both. In some embodiments, the subject is administered an additional treatment, wherein the additional treatment comprises administration of a corticosteroid, an anti-H1 antihistamine, an anti-H2 antihistamine, or any combination thereof. The additional treatment can be administered to the subject 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, or more prior to the administration of the plurality of nanoparticles to the subject. In some embodiments, the additional treatment and the plurality of nanoparticles are administered simultaneously.

Disclosed herein includes a composition, comprising a plurality of nanoparticles complexed with (a) a guide RNA (gRNA) targeting LPA gene (LPA gRNA), and (b) an mRNA encoding Cas9 endonuclease, wherein the gRNA comprises a spacer sequence of any one of SEQ ID NOs: 18-25. Also disclosed herein includes a composition for use in the treatment of a lipoprotein-related diseases, comprising a plurality of nanoparticles complexed with (a) a guide RNA (gRNA) targeting LPA gene (LPA gRNA), and (b) an mRNA encoding Cas9 endonuclease, wherein the gRNA comprises a spacer sequence of any one of SEQ ID NOs: 18-25. The lipoprotein-related disease can be a metabolic disease, a cardiovascular disease, a lipid metabolism disease, or a combination thereof. In some embodiments, the lipoprotein-related disease is calcific aortic valve disease, myocardial infarctions, coronary heart disease, atherosclerosis, thrombosis, stroke, coronary artery disease, familial hyperlipidemia, myocardial infarction, peripheral arterial disease, calcific aortic valve stenosis, or a combination thereof. In some embodiments, the gRNA comprises the spacer sequence of SEQ ID NO: 18. In some embodiments, the gRNA comprises the sequence of SEQ ID NO: 32. In some embodiments, the gRNA comprises the sequence of SEQ ID NO: 11. The Cas9 endonuclease can be S. pyogenes Cas9 endonuclease. In some embodiments, the plurality of nanoparticles are lipid nanoparticles. In some embodiments, the lipid nanoparticles comprise one or more neutral lipids, charged lipids, ionizable lipids, steroids, and polymers conjugated lipids. In some embodiments, the lipid nanoparticles comprise cholesterol, a polyethylene glycol (PEG) lipid, or both. In some embodiments, the composition is a pharmaceutical composition comprising one or more pharmaceutically acceptable excipients.

### BRIEF DESCRIPTION OF THE DRAWINGS

FIG. 1 depicts an exemplary Lipoprotein(a) (Lp(a)) structure.

FIG. 2 depicts a non-limiting exemplary non-human primate (NHP) study design.

FIGS. 3A-B depict plots showing the plasma Lp(a) levels of Group 4 NHPs (FIG. 3A) and Group 5 NHPs (FIG. 3B).

FIGS. 4A-B depict plots showing the percentage change of the plasma Lp(a) levels from baseline of Group 4 NHPs (FIG. 4A) and Group 5 NHPs (FIG. 4B).

FIG. 5 depicts a non-limiting exemplary experimental design. The endpoints include and are not limited to: Hematology: d-14 (i.e., Day −14), d-7 (i.e., Day −7), d7 (i.e., Day 7), d15, d29, d43, d57, d71, and/or d84; Serum Chemistry: d-14, d-7, d2, d4, d7, d10, d15, d29, d43, d57, d71, and/or d84; Coagulation: d-14, d-7, d15, d29, d43, d57, d71, and/or d84; ECG: d-12, d2, d22, and/or d84; Bioanalytical: d-7, d1, d2, d4, d8, d15, d29, d43, d57, d71, and/or d84; Biomarker: d-14, d-7, d8, d15, d29, d43, d57, d71, and/or d84; and Editing in Tissues: tissue list includes reproductive tissues.

FIG. 6 is a graph showing the baseline Lp(a) levels in NHP plasma fourteen days prior to the treatment (Day −14).

FIG. 7 depicts a non-limiting exemplary NHP study design.

FIG. 8A is a graph showing the percentage change of the plasma Lp(a) protein levels from a baseline of NHPs after the CTX320 treatments with three different doses: 0.5 mg/kg, 1.5 mg/kg, and 3 mg/kg in comparison to a control group. FIG. 8B is a graph showing the percentage change of the serum Lp(a) protein levels from a baseline of NHPs after the CTX320 treatments with three different doses: 0.5 mg/kg, 1.5 mg/kg, and 3 mg/kg in comparison to a control group.

FIGS. 9A-C are graphs showing the plasma Lp(a) protein levels of the NHPs before and after treatments with three different doses of CTX320: 0.5 mg/kg (FIG. 9A), 1.5 mg/kg (FIG. 9B) and 3.0 mg/kg (FIG. 9C). FIG. 9D is a graph showing the percentage change of the plasma Lp(a) level from the baseline of the NHPs on Day 29 following the CTX320 treatments.

FIG. 10A is a graph showing the percentage change of the serum Lp(a) protein levels from a baseline of NHPs after the CTX320 treatments with three different doses: 0.5 mg/kg, 1.5 mg/kg, and 3 mg/kg in comparison to a control group. FIG. 10B shows the percentage change of the plasma Lp(a) level from the baseline of the NHPs after about 3-month CTX320 treatment.

FIG. 11 is a plot showing the percentage of LPA gene editing in liver and other organ tissues including spleen, adrenal gland, brain, kidney, lung, epididymis, testes and ovaries.

FIG. 12A-12C are plot showing reproductive tissue editing post-dosing with CTX320.

FIGS. 13A-13B are plots showing the plasma level of LNP component A post treatment.

FIGS. 14A-14B are plots showing the plasma level of LNP component B post treatment.

FIG. 15 shows a non-limiting exemplary design for Phase 1 safety and tolerability clinical study for one or more of the LPA gene-editing nanoparticles described herein (e.g., CTX320).

FIG. 16 is a graph showing the percentage change of the serum Lp(a) protein levels from a baseline of patients after CTX320 treatments. The patients are pretreated with steroids and antihistamine drugs.

FIG. 17 provides plots comparing aspartate aminotransferase (AST), alanine transaminase (ALT), alkaline phosphatase (ALP) and total bilirubin levels in patients pretreated with steroids and antihistamine drugs prior to CTX320 administration (2.0 mg/kg) and in patients without pretreatment and with 1.5 mg/kg and 3 mg/kg CTX320.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

High levels of Lp(a) have been associated with increased risk of cardiovascular disease. Lp(a) levels of greater than 50 mg/dL (e.g., >125 nmol/L) can cause calcific aortic valve disease (CAVD) and cardiovascular disease (CVD). Unlike low-density lipoproteins, Lp(a) levels cannot be modulated by environments, diet, exercise, or existing lipid lowering drugs such as statin, making it a genetically-driven disease risk factor. There are currently no approved medicines that directly target Lp(a). The present disclosure provides methods, compositions, systems and kits for modulating (e.g., decreasing) Lp(a) levels to reduce the risk of cardiovascular diseases and/or treat cardiovascular diseases in subjects in need thereof.

Disclosed herein include methods, compositions and kits for treating a cardiovascular disease or disorder in a subject, e.g., a primate subject. In some embodiments, the method comprises administering to a subject in need thereof a plurality of nanoparticles complexed with (a) a guide RNA (gRNA) targeting LPA gene or a nucleic acid encoding a gRNA that targets LPA gene, and (b) a nucleic acid (e.g., a mRNA) encoding a RNA-guided endonuclease, thereby treating the cardiovascular disease or disorder in the subject.

### Definition

As used herein, the term “about” means plus or minus 5% of the provided value.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

As used herein, a “secondary structure” of a nucleic acid molecule (e.g., an RNA fragment, or a gRNA) refers to the base pairing interactions within the nucleic acid molecule.

As used herein, the term “target DNA” refers to a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

As used herein, the term “Cas endonuclease” or “Cas nuclease” refers to an RNA-guided DNA endonuclease associated with the CRISPR adaptive immunity system.

Unless otherwise indicated “nuclease” and “endonuclease” are used interchangeably herein to refer to an enzyme which possesses endonucleolytic catalytic activity for polynucleotide cleavage.

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

As used herein, the term “donor template” refers to a nucleic acid strand containing exogenous genetic material which can be introduced into a genome (e.g., by a homology directed repair) to result in targeted integration of the exogenous genetic material. In some embodiments, a donor template can have no regions of homology to the targeted location in the DNA and can be integrated by NHEJ-dependent end joining following cleavage at the target site. A donor template can be DNA or RNA, single-stranded or double-stranded, and can be introduced into a cell in linear or circular form.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g. complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e. 100%. For example, the complementary candidate sequence segment is perfectly complementary to the candidate sequence segment, whose sequence can be deducted from the candidate sequence segment using the Watson-Crick base pairing rules.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

As used herein, the terms “transfection” or “infection” refer to the introduction of a nucleic acid into a host cell, such as by contacting the cell with a recombinant MVA virus or a gutless picornaviral particle as described herein.

As used herein, the term “transgene” refers to any nucleotide or DNA sequence that is integrated into one or more chromosomes of a target cell by human intervention. In some embodiment, the transgene comprises a polynucleotide that encodes a protein of interest. The protein-encoding polynucleotide is generally operatively linked to other sequences that are useful for obtaining the desired expression of the gene of interest, such as transcriptional regulatory sequences. In some embodiments, the transgene can additionally comprise a nucleic acid or other molecule(s) that is used to mark the chromosome where it has integrated.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

As used herein, the terms “effective amount” or “pharmaceutically effective amount” or “therapeutically effective amount” refer to an amount sufficient to effect beneficial or desirable biological and/or clinical results.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipient can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. I some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human. In some aspects, the subject can have or is suspected of having a cardiovascular disease and/or has one or more symptoms of a cardiovascular disease. In some aspects, the subject is a human who is diagnosed with a risk of cardiovascular disease at the time of diagnosis or later. In some cases, the diagnosis with a risk of cardiovascular disease can be determined based on the presence of one or more mutations in an endogenous apolipoprotein(a) (LPA) gene or genomic sequence near the LPA gene in the genome that may affect the expression of the apo(a) protein.

The term “plasma level” used herein in the context of a molecule refers to a concentration or an amount of the molecule, e.g., the number of moles or the weight of the molecule present in a given volume of plasma.

High levels of Lp(a) have been associated with increased risk of cardiovascular disease. Lp(a) levels of greater than 50 mg/dL can cause calcific aortic valve disease (CAVD) and cardiovascular disease (CVD). Unlike low-density lipoproteins, Lp(a) levels cannot be modulated by environments, diet, exercise, or existing lipid lowering drugs such as statin, making it a genetically-driven disease risk factor. There is a need for a novel gene therapy that can stably reduce Lp(a) levels over an extended period of time or permanently lowers Lp(a) levels. The present disclosure provides a highly efficient gene editing method and related compositions and kits that directly target the LPA gene or variants thereof to permanently knockout the LPA gene from a genome, thereby permanently reducing the levels of Lp(a) in the blood (e.g., plasma) in a subject. In some embodiments, the methods, compositions, and kits described herein can reduce the plasma Lp(a) levels by at least 20%, at least 30%, at least 40% at least 45%, at least 50%, at least 55%, at least 60%, at least 65%, at least 70%, at least 75%, at least 80%, or greater. In some embodiments, the plasma Lp(a) levels in the subject following carrying out the method is reduced to, or to about, 60 mg/dL, 55 mg/dL, 50 mg/dL, 45 mg/dL, 40 mg/dL, 35 mg/dL, 30 mg/dL, or lower. The gRNA sequences described herein can also significantly minimize the number and frequency of off-target effects, thus reducing the risk of genotoxicity. The methods, compositions, and kits described herein can be used to treat cardiovascular diseases and/or reduce the risk of cardiovascular diseases in a subject.

### Lipoprotein(a) (Lp(a))

Provided herein include vectors, compositions, methods, systems and kits for editing an LPA gene (including, e.g., LPA gene variants associated with increased cardiovascular disease risk and/or increased Lp(a) expression) that encodes the apolipoprotein(a) protein of lipoprotein(a) (Lp(a)) in a cell genome to modulate (e.g., decrease) the expression, function, or activity of the Lp(a) in the cell. The term “LPA gene” as used herein includes the genomic region encompassing the LPA regulatory promoters and enhancer sequences as well as the LPA coding sequence.

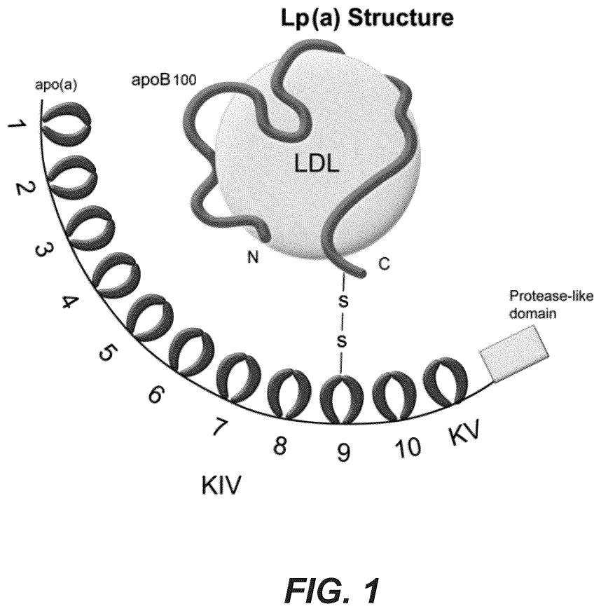

Lp(a) is an atherogenic lipoprotein consisting of the protein apolipoprotein(a) (apo(a)) covalently bound to the apolipoprotein B-100 (apoB) component of a low-density lipoprotein (LDL) particle (see, for example, FIG. 1). The apo(a) protein is encoded by the LPA gene, made in hepatocytes and secreted into circulation. However, while it is known that apo(a) docks to LDL and forms a covalent disulfide bond with apoB to become Lp(a), the precise site of Lp(a) assembly is unknown. This binding of apo(a) to apoB blocks the LDL receptor binding site of apoB and therefore prevents clearance through the LDL receptor pathway. Apo(a) has evolved from the plasminogen gene and contains related protein domains. The apo(a) protein is composed of one kringle V (KV) domain, multiple copies of the kringle IV (KIV) domain, and an inactive protease-like domain, all derived from plasminogen. KIV is broken down into 10 subtypes, with KIVi and KIV3-10 present in 1 copy, and KIV2 present in 1 to greater than 40 copies. The size of apo(a) varies between individual humans and is proportional to the number of copies of KIV2, which is genetically determined. Plasma levels of Lp(a) are inversely correlated to the size of the apo(a) protein and this is thought to be a function of slower secretion of larger isoforms. High plasma level of Lp(a) is an independent risk factor for many cardiovascular diseases, including calcific aortic valve disease, coronary heart disease, atherosclerosis, thrombosis, and stroke (reviewed in Kronenberg, F. (2016). Cardiovasc. Drugs Ther., 30(1):87-100).

The pathogenic mechanisms of Lp(a) is mediated through its pro-atherogenic, pro-inflammatory, and pro-thrombogenic properties. The combination of apo(a) and the LDL components of Lp(a) results in compounding effects on the cardiovascular system. LDL alone can cause immune and inflammatory responses that characterize atherosclerosis through the entry of LDL into vessel walls where the phospholipids become oxidized. Lp(a) circulates and binds to oxidized phospholipids in the plasma, which causes pro-inflammatory responses. Apo(a) itself contains sites that can bind to exposed surfaces on damaged vessel walls, mediating its entry and accumulation at those locations. Small isoforms of apo(a) have been shown to promote thrombosis by inhibiting fibrinolysis.

Plasma levels of Lp(a) have been extensively examined in relation to cardiovascular disease and multiple studies have positively associated high Lp(a) levels to higher risk of cardiovascular disease (reviewed in Kronenberg, F. (2016). Cardiovasc. Drugs Ther., 30(1):87-100). The range of plasma Lp(a) levels in humans can vary by 1000-fold between individuals (e.g., from 0.1 mg/dL to >300 mg/dL or from <30 nmol/L to >400 nmol/L). The variable serum levels are determined genetically by, for example, Apo(a) allele size (number of KIV-2 repeats), 5′ pentanucleotide repeat polymorphism, SNPs in 5′ and other regions of the LPA gene. The Lp(a) level in serum can be detected using ELISA assay or immunoturbidometric analysis as will be understood by a person skilled in the art.

The LPA gene (also known as LP, AK38, and APOA) has a cytogenetic location of 6q25.3-q26 and the genomic coordinate is on chromosome 6 on the reverse strand at position 160531482-160664275. The nucleotide sequence of LPA can be found at NCBI website with the NCBI reference sequence: NC_000006.12. LPA has a NCBI gene ID of 4018, Uniprot ID of P08519 and Ensembl Gene ID of ENSG00000198670.

### Gene Editing

Provided herein includes methods, compositions and kits for editing an LPA gene or variants thereof, thereby reducing the expression level of Lp(a) (e.g., plasma concentrations of Lp(a)) in a subject. Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When an sequence of an endogenous gene is edited, for example by deletion, insertion or substitution of nucleotide(s)/nucleic acid(s), the endogenous gene comprising the affected sequence can be knocked-out or knocked-down due to the sequence alteration. Therefore, targeted editing can be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide can introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Other non-limiting examples of targeted nucleases include naturally-occurring and recombinant nucleases, e.g., CRISPR/Cas9, restriction endonucleases, meganucleases homing endonucleases, and the like.

### CRISPR-Cas Gene Editing System and RNA-Guided Nuclease

In some embodiments, the vectors, compositions, methods, and kits described herein can be used in a gene editing system, such as in a CRISPR-Cas gene editing system, to genetically edit the LPA gene. For example, the CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA) to target the cleavage of DNA. crRNA drives sequence recognition and specificity of the CRISPR-Cas9 complex through Watson-Crick base pairing typically with a 20 nucleotide (nt) sequence in the target DNA. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR). In some embodiments, CRISPR-Cas9 gene editing system comprises an RNA-guided nuclease and one or more guide RNAs targeting one or more target genes.

As described herein, the RNA-guided endonuclease can be naturally-occurring or non-naturally occurring. The Non-limiting Examples of RNA-guided endonuclease include a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, and functional derivatives thereof. In some instances, the RNA-guided endonuclease is a Cas9 endonuclease. The Cas9 endonuclease can be, for example, Streptococcus pyogenes (SpyCas9), Staphylococcus lugdunensis (SluCas9), P. pneumotropica Cas9 (PpCas9), Staphylococcus auricularis Cas9 (SauriCas9), Staphylococcus lugdunensis Cas9 (SlugCas9), Staphylococcus lutrae Cas9 (SlutrCas9) Staphylococcus haemolyticus Cas9 (ShaCas9), Campylobacter jejuni (CjCas9), Staphylococcus aureus (SaCas9), or a variant thereof. In some embodiments, the RNA-guided endonuclease is a variant of Cas9, including but not limited to, a small Cas9, a dead Cas9 (dCas9), and a Cas9 nickase. In some embodiments, a Cas nuclease can comprise a RuvC or RuvC-like nuclease domain (e.g., Cpf1) and/or a HNH or HNH-like nuclease domain (e.g., Cas9). In some embodiments, the Cas9 endonuclease is S. pyogenes Cas9, S. aureus Cas9, N. meningitides Cas9, S. thermophilus Cas9, S. thermophilus 3 Cas9, T. denticola Cas9, or a variant thereof.

The RNA-guided endonuclease can be a small RNA-guided endonuclease. The small RNA-guided endonucleases can be engineered from portions of RNA-guided endonucleases derived from any of the RNA-guided endonucleases described herein and known in the art. The small RNA-guided endonucleases can be, e.g., small Cas endonucleases. In some cases, a small RNA-guided nuclease is shorter than about 1,100 amino acids in length.

The RNA-guided endonuclease can be a mutant RNA-guided endonuclease. For example, the RNA-guided endonuclease can be a mutant of a naturally occurring RNA-guided endonuclease. The mutant RNA-guided endonuclease can also be a mutant RNA-guided endonuclease with altered activity compared to a naturally occurring RNA-guided endonuclease, such as altered endonuclease activity (e.g., altered or abrogated DNA endonuclease activity without substantially diminished binding affinity to DNA). Such modification can allow for the sequence-specific DNA targeting of the mutant RNA-guided endonuclease for the purpose of transcriptional modulation (e.g., activation or repression); epigenetic modification or chromatin modification by methylation, demethylation, acetylation or deacetylation, or any other modifications of DNA binding and/or DNA-modifying proteins known in the art. In some embodiments, the mutant RNA-guided endonuclease has no DNA endonuclease activity.

The RNA-guided endonuclease can be a nickase that cleaves the complementary strand of the target DNA but has reduced ability to cleave the non-complementary strand of the target DNA, or that cleaves the non-complementary strand of the target DNA but has reduced ability to cleave the complementary strand of the target DNA. In some embodiments, the RNA-guided endonuclease has a reduced ability to cleave both the complementary and the non-complementary strands of the target DNA.

In some embodiments, a nucleic acid encoding an RNA-guided endonuclease is administered to the subject. In some embodiments, the nucleic acid can be generated by an in vitro transcription reaction. In some embodiments, generating in vitro transcribed RNA comprises incubating a linear DNA template with an RNA polymerase and a nucleotide mixture under conditions to allow (run-off) RNA in vitro transcription. The nucleotide mixture can be part of an in vitro transcription mix (IVT-mix). In some embodiments, the RNA polymerase is a T7 RNA polymerase. The nucleotide mixture used in RNA in vitro transcription can additionally contain modified nucleotides as defined below. In some embodiments, the nucleotide mixture (e.g., the fraction of each nucleotide in the mixture) used for RNA in vitro transcription reactions can be optimized for the given RNA sequence (optimized NTP mix). Such methods are described, for example in WO2015/188933. RNA obtained by a process using an optimized NTP mix is, in some embodiments, characterized by reduced immune stimulatory properties.

In some embodiments, the nucleotide mixture comprises non-modified ribonucleoside triphosphates (NTPs) GTP, ATP, CTP and UTP. In some embodiments, the in vitro transcription can include the presence of at least one cap analog, e.g., a cap1 trinucleotide cap analog, m7G(5′)ppp(5′)(2′OMeA)pG or m7G(5′)ppp(5′)(2′OMeG)pG, m7G(5′)ppp(5′)(2′OMeA)pG or rn7(3′OMeG)(5′)ppp(5′)(2′OMeA)pG. In some embodiments, a 5′-cap structure is formed via enzymatic capping using capping enzymes (e.g. vaccinia virus capping enzymes and/or cap-dependent 2′-O-methyltransferases) to generate cap0 or cap1 or cap2 structures. The 5′-cap structure (cap0 or cap1) may also be added using immobilized capping enzymes and/or cap-dependent 2′-O-methyltransferases using methods and means disclosed in WO2016/193226. In some embodiments, a part or all of at least one (ribo)nucleoside triphosphate is replaced by a modified nucleoside triphosphate. In some embodiments, the modified nucleoside triphosphate comprises pseudouridine (ψ), N1-methylpseudouridine (m1 ψ), 5-methylcytosine, or 5-methoxyuridine. In some embodiments, uracil nucleotides in the nucleotide mixture are replaced (either partially or completely) by pseudouridine (ψ) and/or N1-methylpseudouridine (m1 ψ) to obtain a modified RNA. In some embodiments, the chemically modified nucleotide is pseudouridine (ψ). In some embodiments the chemically modified nucleotide is N1-methylpseudouridine (m1ψ). In some embodiments, the nucleotide mixture comprises at least one modified nucleotide and/or at least one nucleotide analogue or nucleotide derivative for incorporation into an RNA. For example, the modified nucleotide as defined herein can include nucleotide analogs/modifications, e.g., backbone modifications, sugar modifications or base modifications. A backbone modification can comprise a modification, in which phosphates of the backbone of the nucleotides are chemically modified. A sugar modification can comprise a chemical modification of the sugar of the nucleotides. Furthermore, a base modification can comprise a chemical modification of the base moiety of the nucleotides. In this context nucleotide analogs or modifications can comprise nucleotide analogs which are applicable for transcription and/or translation. In some embodiments the nucleotide mixture comprises least one modified nucleotide and/or at least one nucleotide analogues is selected from a backbone modified nucleotide, a sugar modified nucleotide and/or a base modified nucleotide, or any combination thereof.

The modified nucleosides and nucleotides, which may be included in the nucleotide mixture and incorporated into the RNA can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂₀)ₙCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g., NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

The phosphate backbone can further be modified in the modified nucleosides and nucleotides, which can be included in the nucleotide mixture and incorporated into a modified in vitro transcribed RNA. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

A nucleotide as described herein can be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications includes an amino group, a thiol group, an alkyl group, or a halo group.

The nucleotide analogues/modifications can comprise 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, puromycin-5′-triphosphate, xanthosine-5′-triphosphate. Base-modified nucleotides can comprise 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, or 7-deaza-adenosine.

At least one modified nucleotide and/or the at least one nucleotide analog described herein can comprise 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, or 5-(isopentenylaminomethyl)-2′-O-methyluridine.

In some embodiments, chemical modifications comprise pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, 2′-O-methyluridine, or a combination thereof.

In some embodiments, 100% of the uracil in the coding sequence can have a chemical modification. In some embodiments, a chemical modification is in the 5′-position of the uracil. In some embodiments, 100% of the uracil in the coding sequence (cds) of the RNA can have a chemical modification, e.g., a chemical modification that is in the 5′-position of the uracil. In some embodiments, at least 10%, 20%, 30%, 40%, 50%, 60%, 70%, 80%, or 90% of the uracil nucleotides in the cds have a chemical modification, e.g., a chemical modification that is in the 5-position of said uracil nucleotides. Such modifications may reduce the stimulation of the innate immune system (after in vivo administration of the RNA comprising such a modified nucleotide).

The terms “cds” or “coding sequence” or “coding region” as used herein will be recognized and understood by the person of ordinary skill in the art, and are e.g., can refer to a sequence of several nucleotide triplets, which may be translated into a peptide or protein. The cds of the RNA may comprise at least one modified nucleotide, wherein said at least one modified nucleotide may be selected from pseudouridine (ψ), N1-methylpseudouridine (m1ψ), 5-methylcytosine, and 5-methoxyuridine.

As used herein, the terms “modified nucleotides” or “chemically modified nucleotides” can refer to all potential natural and non-natural chemical modifications of the building blocks of an RNA, namely the ribonucleotides A, G, C, and U.

In some embodiments the nucleotide mixture in an in vitro transcription reaction comprises a cap analog. Accordingly, in some embodiments the cap analog is a cap0, cap1, cap2, a modified cap0 or a modified cap1 analog, or a cap1 analog as described below. The term “cap analog” or “5′-cap structure” used herein can refer to the 5′ structure of the RNA, particularly a guanine nucleotide, positioned at the 5′-end of an RNA, e.g., an mRNA. In some embodiments, the 5′-cap structure is connected via a 5′-5′-triphosphate linkage to the RNA. In some embodiments, a “5′-cap structure” or a “cap analogue” is not considered to be a “modified nucleotide” or “chemically modified nucleotides”. 5′-cap structures which may be suitable include cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g. phosphothioate modARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure can be formed in chemical RNA synthesis, using capping enzymes, or in RNA in vitro transcription (co-transcriptional capping) using cap analogs. The term “cap analog” as used herein can refer to a non-polymerizable di-nucleotide or tri-nucleotide that has cap functionality in that it facilitates translation or localization, and/or prevents degradation of the RNA when incorporated at the 5′-end of the RNA. Non-polymerizable means that the cap analogue will be incorporated only at the 5′-terminus because it does not have a 5′ triphosphate and therefore cannot be extended in the 3′-direction by a template-dependent polymerase, (e.g., a DNA-dependent RNA polymerase). Examples of cap analogues include m7GpppG, m7GpppA, m7GpppC; unmethylated cap analogues (e.g., GpppG); dimethylated cap analogue (e.g., m2,7GpppG), trimethylated cap analogue (e.g. m2,2,7GpppG), dimethylated symmetrical cap analogues (e.g. m7Gpppm7G), or anti reverse cap analogues (e.g., ARCA; m7,2′OmeGpppG, m7,2′dGpppG, m7,3′OmeGpppG, m7,3′dGpppG and their tetraphosphate derivatives). Further cap analogues have been described previously, e.g., WO2008/016473, WO2008/157688, WO2009/149253, WO2011/015347, and WO2013/059475. Further suitable cap analogues in that context are described in, e.g., WO2017/066793, WO2017/066781, WO2017/066791, WO2017/066789, WO2017/053297, WO2017/066782, WO2018/075827 and WO2017/066797 wherein the disclosures relating to cap analogues are incorporated herewith by reference.

In some embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. For example, any cap analog derivable from the structure disclosed in claim 1-5 of WO2017/053297 may be suitably used to co-transcriptionally generate a cap1 structure. In some embodiments, any cap analog derivable from the structure described in WO2018/075827 can be suitably used to co-transcriptionally generate a cap1 structure. In some embodiments, the cap1 analog is a cap1 trinucleotide cap analog. In some embodiments, the cap1 structure of the in vitro transcribed RNA is formed using co-transcriptional capping using tri-nucleotide cap analog m7G(5′)ppp(5′)(2′OMeA)pG or m7G(5′)ppp(5′)(2′OMeG)pG. In some embodiments, the cap1 analog is m7G(5′)ppp(5′)(2′OMeA)pG.

In some embodiments, the RNA (e.g., mRNA) comprises a 5′-cap structure, e.g., a cap1 structure. In some embodiments, the 5′ cap structure can improve stability and/or expression of the mRNA. A cap1 structure comprising mRNA (produced by, e.g., in vitro transcription) has several advantageous features including an increased translation efficiency and a reduced stimulation of the innate immune system. In some embodiments, the in vitro transcribed RNA comprises at least one coding sequence encoding at least one peptide or protein. In some embodiments, the protein is an RNA-guided endonuclease. In some embodiments, the RNA-guided endonuclease is Cas9 or a derivative thereof.

The present disclosure provides optimized mRNAs encoding an S. pyogenes Cas9 endonuclease (“SpCas9 mRNA”), and which optionally include chemically modified nucleotides, that provide effective genome editing of a target cell population when administered with one or more gRNAs. In some embodiments, the disclosure provides an mRNA comprising (i) a 5′ untranslated region (UTR); (ii) an open reading frame (ORF) comprising a nucleotide sequence that encodes a site-directed endonuclease; and (iii) a 3′ untranslated region (UTR). In some embodiments, the site-directed endonuclease is a Cas nuclease. In some embodiments, the Cas nuclease is a Cas9 polypeptide. In some embodiments, the Cas9 polypeptide is a Streptococcus pyogenes-derived Cas9 (SpCas9) polypeptide. In some embodiments, the ORF further comprises one or more nucleotide sequences encoding a nuclear localization signal, such as one described herein. In some embodiments, the ORF comprises a nucleotide sequence encoding a site-directed endonuclease, such as a SpCas9 polypeptide and at least one NLS that is a nucleoplasmin and/or SV40 NLS. In some embodiments, the ORF comprises a nucleotide sequence encoding an N-terminal and/or C-terminal NLS operably-linked to a site-directed endonuclease, such as a SpCas9 polypeptide. In some embodiments the ORF comprises a nucleotide sequence encoding an N-terminal SV40 NLS operably-linked to a site-directed endonuclease, such as a SpCas9 polypeptide, and a C-terminal nucleoplasmin NLS operably-linked to the site-directed endonuclease, such as the SpCas9 polypeptide.

In some embodiments, the mRNA can comprise at least one chemically modified nucleoside and/or nucleotide. In some embodiments, the chemically modified nucleoside is selected from pseudouridine, N1-methylpseudouridine, and 5-methoxyuridine. In some embodiments, the chemically modified nucleoside is N1-methylpseudouridine (e.g., 1-methylpseudouridine). In some embodiments, at least about 80% or more (e.g., about 85%, 90%, 91%, 92%, 93%, 94%, 95%, 96%, 97%, 98%, 99%) of uridines in the mRNA are modified or replaced with N1-methylpseudouridine. In some embodiments, 100% of the uridines (e.g., uracils) in the mRNA are modified or replaced with N1-methylpseudouridine. In some embodiments, two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800 or more) of the uridine or uracil residues of the mRNA are N1-methylpseudouridine.

Guide RNAs (gRNAs)

In some embodiments, the CRISPR/Cas-mediated gene editing system used to genetically edit a LPA gene comprises a genome-targeting nucleic acid (e.g., a guide RNA) that can direct the activities of a RNA-guided endonuclease to a specific target sequence within the LPA gene. A guide RNA comprises at least a spacer sequence that hybridizes to a target nucleic acid sequence of interest, and a CRISPR repeat sequence. The gRNA can be a single-molecule guide RNA or a double-molecule guide RNA. The RNA-guided endonuclease can be, for example a Cas endonuclease, including Cas9 endonuclease. The Cas9 endonuclease can be, for example, a SpyCas9, a SaCas9, or a SluCas9 endonuclease. In some embodiments, the RNA-endonuclease is a Cas9 variant. In some embodiments, the RNA-guided endonuclease is a small RNA-guided endonuclease. In some embodiments, the RNA-guided endonuclease is a small Cas endonuclease.

In some embodiments, the gRNA comprise 5′ to 3′: a crRNA and a tracrRNA, wherein the crRNA and tracrRNA hybridize to form a duplex. In some embodiments, the crRNA comprises a spacer sequence capable of targeting a target sequence in a target nucleic acid (e.g., genomic DNA molecule) and a crRNA repeat sequence. In some embodiments, the tracrRNA comprises a tracrRNA anti-repeat sequence and a 3′ tracrRNA sequence. In some embodiments, the 3′ end of the crRNA repeat sequence is linked to the 5′ end of the tracrRNA anti-repeat sequence, e.g., by a tetraloop, wherein the crRNA repeat sequence and the tracrRNA anti-repeat sequence hybridize to form the sgRNA. In some embodiments, the sgRNA comprises 5′ to 3′: a spacer sequence, a crRNA repeat sequence, a tetraloop, a tracrRNA anti-repeat sequence, and a 3′ tracrRNA sequence. In some embodiments, the sgRNA comprise a 5′ spacer extension sequence. In some embodiments, the sgRNA comprise a 3′ tracrRNA extension sequence. The 3′ tracrRNA can comprise, or consist of, one or more stem loops, for example one, two, three, or more stem loops.

In some embodiments, the invariable sequence of the sgRNA comprises the nucleotide sequence of GUUUUAGAGCUAGAAAUAGCAAGUUAAAAUAAGGCUAGUCCGUUAUCAACUUGA AAAAGUGGCACCGAGUCGGUGCUUUU (SEQ ID NO: 1), or a nucleotide sequence having up to 1, 2, 3, 4, 5, 6, 7, 8, 9, or 10 nucleotide deletions, insertions, or substitutions relative to SEQ ID NO: 1. In some embodiments, the sgRNA is for use with a SpCa9 or a SpyCas9 endonuclease.

The guide RNA disclosed herein can target any sequence of interest via the spacer sequence in the crRNA. A spacer sequence in a gRNA is a sequence (e.g., a 20 nucleotide sequence) that defines the target sequence (e.g., a DNA target sequences, such as a genomic target sequence) of a target gene of interest (e.g., LPA gene). In some embodiments, the spacer sequence range from 15 to 30 nucleotides. For example, the spacer sequence can be, can be about, can be at least, or can be at most 10, 15, 16, 17, 18, 19, 29, 21, 22, 23, 24, 25, 26, 27, 28, 29, 30, 40, 50, or a number or a range between any of these values, of nucleotides in length. In some embodiments, a spacer sequence contains 20 nucleotides. In some embodiments, the gRNA is capable of hybridizing to the forward strand of the target dsDNA. In some embodiments, the gRNA is capable of hybridizing to the reverse strand of the target dsDNA.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. The spacer of a gRNA interacts with a target nucleic acid of interest in a sequence-specific manner via hybridization (i.e., base pairing). The nucleotide sequence of the spacer thus varies depending on the target sequence of the target nucleic acid of interest. In some embodiments, the target sequence of the LPA gene is within exon 3 of the LPA gene.

In a CRISPR/Cas system used herein, the spacer sequence is designed to hybridize to a region of the target nucleic acid that is located 5′ of a PAM recognizable by a Cas9 enzyme used in the system. The spacer can perfectly match the target sequence or can have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNRG-3′ (SEQ ID NO: 2), the target nucleic acid can be the sequence that corresponds to the Ns, wherein N can be any nucleotide, and the underlined NRG sequence (R is G or A) is the S. pyogenes PAM. In some embodiments, the PAM sequence used in the compositions and methods of the present disclosure as a sequence recognized by SpCas9 is NGG, wherein N can be A, T, C or G.

The percent complementarity between the spacer sequence and the target nucleic acid can be about, at least, at least about, at most or at most about 30%, 40%, 50%, 60%, 65%, 70%, 75%, 80%, 85%, 90%, 95%, 97%, 98%, 99%, or 100%. In some embodiments, the spacer sequence of the guide RNA and the target nucleic acid in the target gene is 100% complementary In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In other embodiments, the spacer sequence of the guide RNA and the target sequence in the target gene can contain up to 10 mismatches, e.g., up to 9, up to 8, up to 7, up to 6, up to 5, up to 4, up to 3, up to 2, or up to 1 mismatch.

The LPA gRNA can target a target sequence within exon 3 of the LPA gene. In some embodiments, the LPA gRNA comprises a spacer sequence corresponding to any one of the target sequences set forth in SEQ ID NOs: 3-10 listed in Table 1. In some embodiments, the LPA gRNA comprises a spacer sequence selected from SEQ ID NOs: 18-25 listed in Table 1.

TABLE 1
Exemplary target/spacer sequences

| | Target/Spacer | | |
|---|---|---|---|
| Guide | Sequence | PAM | SEQ ID NO |
| T4 | GATTAATGAC | GGG | 3 (Target) |
| | ATACGCATTT | | |
| | (Target) | | |
| | GAUUAAUGAC | | 18 (Spacer) |
| | AUACGCAUUU | | |
| | (Spacer) | | |
| T1 | TGGACCACAT | AGG | 4 (Target) |
| (T5513) | GGCTTTGCTC | | |
| | (Target) | | |
| | UGGACCACAU | | 19 (Spacer) |
| | GGCUUUGCUC | | |
| | (Spacer) | | |
| T2 | TGAGCAAAGC | AGG | 5 (Target) |
| (T13789) | CATGTGGTCC | | |
| | (Target) | | |
| | UGAGCAAAGC | | 20 (Spacer) |
| | CAUGTGGUCC | | |
| | (Spacer) | | |
| T10762 | GTGGTCCTAT | TGG | 6 (Target) |
| | TATGTTGATG | | |
| | (Target) | | |
| | GUGGUCCUAU | | 21 (Spacer) |
| | UAUGUUGAUG | | |
| | (Spacer) | | |
| T3893 | CATAGATGAC | AGG | 7 (Target) |
| | CAAGCTTGGC | | |
| | (Target) | | |
| | CAUAGAUGAC | | 22 (Spacer) |
| | CAAGCUUGGC | | |
| | (Spacer) | | |
| T3894 | CATAGATGAC | AGG | 8 (Target) |
| | CAAGCTTGGC | | |
| | (Target) | | |
| | CAUAGAUGAC | | 23 (Spacer) |
| | CAAGCUUGGC | | |
| | (Spacer) | | |
| T14467 | AGCACCTGAG | TGG | 9 (Target) |
| | CAAAGCCATG | | |
| | (Target) | | |
| | AGCACCUGAG | | 24 (Spacer) |
| | CAAAGCCAUG | | |
| | (Spacer) | | |
| T5 | AGATTAATGA | TGG | 10 (Target) |
| (T1530) | CATACGCATT | | |
| | (Target) | | |
| | AGAUUAAUGA | | 25 (Spacer) |
| | CAUACGCAUU | | |
| | (Spacer) | | |

In some embodiments, the gRNA comprises a spacer sequence corresponding to any one of the target sequences set forth in SEQ ID NOs: 3-10 or a variant thereof. In some embodiments, the gRNA comprises a spacer sequence corresponding to any one of the target sequences set forth in SEQ ID NOs: 3-10. In some embodiments, the gRNA comprises a spacer sequence having one, two or three mismatches to a RNA sequence corresponding to any one of the target sequences set forth in SEQ ID NOs: 3-10. In some embodiments, the gRNA comprises a spacer sequence set forth in SEQ ID NOs: 18-25 or a variant thereof. In some embodiments, the gRNA comprises a spacer sequence selected from SEQ ID NOs: 18-25. In some embodiments, the gRNA comprises a spacer sequence having one, two or three mismatches to a sequence selected from SEQ ID NOs: 18-25. In some embodiments, the gRNA is a sgRNA.

In some embodiments, two gRNAs comprising spacers complementary to a target sequence of the LPA gene are provided to a cell. In some embodiments, the gRNAs are any two gRNAs comprising spacers corresponding to any one of the target sequences set forth in SEQ ID NOs: 3-10 or a variant thereof. In some embodiments, one or both of the two gRNAs comprise a spacer sequence corresponding to any one of the target sequences set forth in SEQ ID NOs: 3-10. In some embodiments, one or both of the two gRNA comprises a spacer sequence having one, two or three mismatches to a RNA sequence corresponding to any one of the target sequences set forth in SEQ ID NOs: 3-10. In some embodiments, one or both of the two gRNAs comprise a spacer sequence set forth in SEQ ID NOs: 18-25 or a variant thereof. In some embodiments, one or both of the two gRNAs comprise a spacer sequence selected from SEQ ID NOs: 18-25. In some embodiments, one or both of the two gRNAs comprise a spacer sequence having one, two or three mismatches to a sequence selected from SEQ ID NOs: 18-25.

In some embodiments, the gRNAs comprises a first gRNA comprising a spacer having a sequence of SEQ ID NO: 18 or a sequence having at least 85% (e.g., at least 90% or at least 95%) homology to SEQ ID NO: 18 and a second gRNA comprising a spacer having a sequence of any one of SEQ ID NOs: 19-25 or a sequence having at least 85% (e.g., at least 90% or at least 95%) homology to any one of SEQ ID NOs: 19-25. In some embodiments, the gRNAs comprises a first gRNA comprising a spacer having a sequence of SEQ ID NO: 18 and a second gRNA comprising a spacer having a sequence of any one of SEQ ID NOs: 19-25.

In some embodiments, the gRNA comprises a spacer sequence set forth in SEQ ID NO: 18 or a sequence having about, at least, at least about 80%, 85%, 90%, 95%, 97%, 98%, 99%, or 100% homology to SEQ ID NO: 18. In some embodiments, the gRNA comprises a spacer sequence having no more than 3 mismatches (for example, 1 or 2 mismatches) relative to SEQ ID NO: 18. In some embodiments, the gRNA comprises a spacer sequence of SEQ ID NO: 18.

In some embodiments, the LPA gRNA comprises a spacer sequence corresponding to any one of the target sequences set forth in SEQ ID NOs: 12-17 listed in Table 2 below. In some embodiments, the LPA gRNA comprises a spacer sequence selected from SEQ ID NOs: 26-31 listed in Table 2.

TABLE 2
Exemplary guide RNA target/spacer sequences

| Region of | | Guide RNA Target/ | |
|---|---|---|---|
| gene | Guide | Spacer Sequence | SEQ ID NO |
| Kringle | T146 | GGATCCCTCG | 12 (Target) |
| IV | | TATAACAATA | |
| Exon | | (Target) | |
| 4 | | GGAUCCCUCG | 26 (Spacer) |
| | | UAUAACAAUA | |
| | | (Spacer) | |
| Kringle | T146 | GGATCCCTCG | 12 (Target) |
| IV | | TATAACAATA | |
| Exon | | (Target) | |
| 18 | | GGAUCCCUCG | 26 (Spacer) |
| | | UAUAACAAUA | |
| | | (Spacer) | |
| Regulatory | T4110 | TGGACTACAT | 13 (Target) |
| | | AGTTGTGTGA | |
| | | (Target) | |
| | | UGGACUACAU | 27 (Spacer) |
| | | AGUUGUGUGA | |
| | | (Spacer) | |
| Regulatory | T7918 | TTCAATCCTC | 14 (Target) |
| | | TTGTCACCTG | |
| | | (Target) | |
| | | UUCAAUCCUC | 28 (Spacer) |
| | | UUGUCACCUG | |
| | | (Spacer) | |
| Regulatory | T14868 | CTGCCAGACT | 15 (Target) |
| | | CTCTGAACCC | |
| | | (Target) | |
| | | CUGCCAGACU | 29 (Spacer) |
| | | CUCUGAACCC | |
| | | (Spacer) | |
| Regulatory | T524 | AAAACCTACC | 16 (Target) |
| | | GCAAGTTGCC | |
| | | (Target) | |
| | | AAAACCUACC | 30 (Spacer) |
| | | GCAAGUUGCC | |
| | | (Spacer) | |
| Regulatory | T221 | GACATCTCTA | 17 (Target) |
| | | TGTCGGCCAC | |
| | | (Target) | |
| | | GACAUCUCUA | 31 (Spacer) |
| | | UGUCGGCCAC | |
| | | (Spacer) | |

In some embodiments, the gRNA is a chemically modified gRNA. Various types of RNA modifications can be introduced to the gRNAs to enhance stability, reduce the likelihood or degree of innate immune response, and/or enhance other attributes as described in the art. The gRNAs described herein can comprise one or more modifications including internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

The chemically-modified gRNA can comprise one or more phosphorothioated 2′-O-methyl nucleotides at the 3′ end and/or the 5′ end of the gRNA. In some embodiments, the chemically-modified gRNA comprises phosphorothioated 2′-O-methyl nucleotides at the 3′ end of the gRNA. In some embodiments, the chemically-modified gRNA comprises phosphorothioated 2′-O-methyl nucleotides at the 5′ end of the gRNA. In some embodiments, the chemically-modified gRNA comprises three or four phosphorothioated 2′-O-methyl nucleotides at the 3′ end and/or three or four at the 5′ end of the gRNA. In some embodiments, any one of SEQ ID NOs: 18-25 and 26-31 can be chemically modified to have one, two three or four phosphorothioated 2′-O-methyl nucleotides at the 3′ end of the gRNA; one, two or three phosphorothioated 2′-O-methyl nucleotides at the 5′ end of the gRNA, or a combination thereof. The number and position of the phosphorothioate linkages can vary. In some embodiments, the linkage can be between the first and second, the second and third, the third and fourth position, fourth and fifth, fifth and sixth, sixth and seventh, seventh and eighth, eighth and ninth, ninth or tenth, or further, position from the 5′ end of the gRNA. In some embodiments, the linkage can be between the first and second, the second and third, the third and fourth position, fourth and fifth, fifth and sixth, sixth and seventh, seventh and eighth, eighth and ninth, ninth or tenth, or further, position from the 3′ end of the gRNA. In some embodiments, the chemically modified gRNA has the sequence of any one of the gRNAs described herein (e.g., SEQ ID NO: 32), and with one or more chemically modified nucleotides described herein and/or one or more of phosphorothioate linkages. As an example, a chemically modified gRNA is SEQ ID NO: 11.

In some embodiments, the nucleotide analogues/modifications can comprise 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, puromycin-5′-triphosphate, or xanthosine-5′-triphosphate. Base-modified nucleotides can comprise 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, or 7-deaza-adenosine.

At least one modified nucleotide and/or the at least one nucleotide analog can comprise 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, or 5-(isopentenylaminomethyl)-2′-O-methyluridine.

In some embodiments, chemical modifications comprise pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyluridine. In some embodiments, the modification comprises a 2′-O-methyluridine (2′OMe-rU), a 2-O-methylcytidine (2′OMe-rC), 2′-O-methyladenosine (2′OMe-rA), or 2′-O-methylguanosine (2′OMe-rG).

The gRNA can comprise any number of modified nucleotides. For example, the percentage of nucleotides in a gRNA molecule that are modified can be, can be at least, can be about, or can be at least about 1%, 2%, 3%, 4%, 5%, 6%, 7%, 8%, 9%10%1%, i1%, i2%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%.

As an example, SEQ ID NO: 3 can be chemically modified as: “g*a*u* UAA UGA CAU ACG CAU UU” (Ts are converted to Us), in which u=2′OMe-rU; a=2′OMe-rA; g =2′OMe-rG; *=Thiolated Phosphate. In some embodiments, the LPA gRNA comprises chemically modified SEQ ID NO: 18, for example “g*a*u* UAA UGA CAU ACG CAU UU” in which u=2′OMe-rU; a=2′OMe-rA; g=2′OMe-rG; *=Thiolated Phosphate.

In some embodiments, the gRNA comprises, or consists of, GAUUAAUGACAUACGCAUUUGUUUUAGAGCUAGAAAUAGCAAGUUAAAAUAAGG CUAGUCCGUUAUCAACUUGAAAAAGUGGCACCGAGUCGGUGCUUUU (SEQ ID NO: 32). One or more of the nucleotides of the gRNA (e.g., SEQ ID NO: 32) can be modified nucleotide(s), for example one or more Us of the gRNA (e.g., SEQ ID NO: 32) can 2′OMe-rU, one or more As of the gRNA (e.g., SEQ ID NO: 32) can 2′OMe-rA, one or more Cs of the gRNA (e.g., SEQ ID NO: 32) can 2′OMe-rC, and one or more Gs of the gRNA (e.g., SEQ ID NO: 32) can 2′OMe-rG. In one or more positions of the gRNA (e.g., SEQ ID NO: 32) can be modified, e.g., Thiolated Phosphate. In some embodiments, the gRNA comprises, or consists of, the sequence of SEQ ID NO: 11: 5′-g*a*u* UAA UGA CAU ACG CAU UUG UUU UAG Agc uag aaa uag cAA GUU AAA AUA AGG CUA GUC CGU UAU Caa cuu gaa aaa gug gca ccg agu cgg ugc u*u*u*u-3′, wherein, u=2′OMe-rU; a=2′OMe-rA; c=2′OMe-rC; g=2′OMe-rG; *=Thiolated Phosphate. The underlined sequence corresponds to the spacer.

In some embodiments, more than one guide RNA can be used with a CRISPR/Cas nuclease system. Each guide RNA can contain a different targeting sequence, such that the CRISPR/Cas system cleaves more than one target nucleic acid. In some embodiments, one or more guide RNAs can have the same or differing properties such as activity or stability within the Cas9 RNP complex. Where more than one guide RNA is used, each guide RNA can be encoded on the same or on different vectors.

gRNAs described herein can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Polynucleotides constructs and vectors can be used to in vitro transcribe a gRNA described herein.

### Methods of Editing LPA

Provided herein includes a method of using genome editing to edit LPA by functionally knocking out or reducing the expression of the LPA gene in the genome of a cell. The method can be used to treat a subject with a lipoprotein-related disease or disorder, e.g., a patient with a cardiovascular disease. In some embodiments, the method comprises administering to a subject (e.g., a primate subject) a plurality of nanoparticles complexed with (a) a guide RNA (gRNA) or a nucleic acid encoding a gRNA that targets LPA gene, and (b) a nucleic acid encoding a RNA-guided endonuclease, thereby treating the cardiovascular disease in the primate subject.

The subject can be administered with the plurality of nanoparticles two or more times, for example twice, for the treatment. Two administration of the nanoparticles to the subject can be separated by a suitable time period, for example one week, two weeks, three weeks, four weeks, five weeks, six weeks, seven weeks, eight weeks, three months, four months, five months, six months, a year, eighteen months, two years, three years, five years, ten years, fifteen years, or more. In some embodiments, two of the two or more administrations are about two weeks to about two months apart, for example about three weeks. In some embodiments, each two of the two or more administrations are about two weeks to about two months apart, for example about three weeks. The suitable time period between two administrations can be the same as or different from the suitable time period between another two administrations. In some embodiments, the method described herein comprises administration of a single dose of the plurality of nanoparticles to the subject in a provided period of time, for example, one year, two years, three years, five years, six years, eight years, ten years, fifteen years, twenty years, or longer. In some embodiments, the method described herein comprises administration of a single dose of the plurality of nanoparticles to a subject in the subject's life time. In some embodiments, the method described herein comprises administration of a single dose of the plurality of nanoparticles to the subject. In some embodiments, the plurality of nanoparticles is administered to the subject at a dose of about 0.01-5 mg/kg, for example 0.05-2 mg/kg, 0.5-3 mg/kg or 0.1-1 mg/kg, gRNA per administration. In some embodiments, the plurality of nanoparticles is administered to the subject at a dose of about 0.01-5 mg/kg, for example 0.05-2 mg/kg, 0.5-3 mg/kg or 0.1-1 mg/kg, total nucleic acids (i.e., the total of the LPA gRNA and RNA encoding the RNA-guided endonuclease (e.g., Cas9 mRNA)) per administration.

In some embodiments, the gRNA targets within or near a coding sequence in the LPA gene. In some embodiments, the gRNA targets any one of the exons of the LPA gene. In some embodiments, the gRNA targets a sequence within exon 3 of the LPA gene. The gRNA can comprise a spacer sequence complementary to a target sequence within exon 3 of the LPA gene. In some embodiments, the spacer(s) are complementary to a sequence within or near (for example, within any of 1, 2, 3, 4, 5, 6, 7, 8, 9, 10, 15, 20, 25, 30, 35, 40, 45, 50, 55, 60, 65, 70, 75, 80, 85, 90, 95, 100 or more bases from) exon 3 of the LPA gene. The complementarity between the spacer of the gRNA and the target sequence in the LPA gene can be perfect or imperfect. In some embodiments, the complementarity can be at least 70%, 80%, 90%, 100% or a number or a range between any two of these values. In some embodiments, the complementarity is perfect, i.e., 100%.

In some embodiments, the gRNA comprises a spacer sequence from any one of SEQ ID NOs: 18-25 or a sequence having at least or at least about 80%, 85%, 90%, 95%, 97%, 98%, 99%, or 100% homology to any one of SEQ ID NOs: 18-25. In some embodiments, the gRNA comprises a spacer sequence from any one of SEQ ID NOs: 18-25 or a sequence having one, two or three mismatches as relative to any one of SEQ ID NOs: 18-25.

In some embodiments, the gRNA comprises the sequence of SEQ ID NO: 11 or a sequence having about, at least, or at least about 80%, 85%, 90%, 95%, 97%, 98%, 99%, or 100% sequence homology to SEQ ID NO: 11. In some embodiments, the gRNA comprises e sequence of SEQ ID NO: 11.

The gRNAs used herein can enhance on-target activity while significantly reducing potential off-target effects (i.e., cleaving genomic DNA at undesired locations other than LPA gene). In some embodiments, the off-target binding is reduced by about, at least or at least about 80%, 85%, 90%, 95%, 98%, 99% or 100%.

The RNA-guided endonuclease can be a Cas endonuclease described herein or known in the art. The Cas endonuclease can be naturally-occurring or non-naturally-occurring (e.g., recombinant or with mutations). In some embodiments, the Cas endonuclease is a Cas9 endonuclease or a variant thereof. In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a functional derivative thereof. In some embodiments, the DNA endonuclease is a Cas9 endonuclease or a variant thereof. In some embodiments, the Cas9 endonuclease is from Streptococcus pyogenes (SpyCas9). In some embodiments, the Cas9 endonuclease is from Staphylococcus lugdunensis (SluCas9).

In some embodiments, the genetic modification of the LPA gene results in a significantly reduced blood or plasma Lp(a) level in the subject (e.g., mammal, NHP, a human subject). In some embodiments, the plasma Lp(a) level is reduced by 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% or a number or a range between any two of these values. In some embodiments, the reduction in the plasma Lp(a) level is about, at least, or at least about 50%, 55%, 60%, 65%, 70%, 75%, 80%, 85%, 90%, 95%, 98%, 99%, 100% or a number or a range between any two of these values.

In some embodiments, the plasma Lp(a) level in a genetically modified subject (e.g., mammal, NHP, a human subject) is about, less than or less than about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, as compared to a corresponding unmodified mammal.

In some embodiments, the reduction is relative to the LPA expression or the concentration of LPA protein in the plasma of the subject (e.g., e.g., mammal, NHP, a human subject) prior to being administered with the plurality nanoparticles. In some embodiments, the reduction is relative to the LPA expression or the concentration of LPA protein in one or more untreated subjects. In some embodiments, the reduction is relative to a reference level of LPA expression or the concentration of LPA protein of healthy and/or unmodified subjects.

In some embodiments, the plasma Lp(a) level following carrying out the method is reduced to about 50 mg/dL or lower (e.g., about, at most, or at most about 40 mg/dL, 30 mg/dL, 20 mg/dL, or lower). In some embodiments, the plasma Lp(a) level following carrying out the method is reduced to about 40 mg/dL or lower. In some embodiments, the plasma Lp(a) level following carrying out the method is reduced to about 30 mg/dL or lower. In some embodiments, the plasma Lp(a) level in following carrying out the method is reduced to about 20 mg/dL or lower.

### Pharmaceutical Compositions and Therapeutic Applications

Provided herein also includes a pharmaceutical composition for carrying out the methods disclosed herein. A composition can include one or more gRNA(s), a RNA-guided endonuclease or a nucleotide sequence encoding the RNA-guided endonuclease described herein. In some embodiments, the composition can further comprises a polynucleotide to be inserted (e.g., a donor template) to affect the desired genetic modification of the methods disclosed herein.

In some embodiments, the one or more gRNA(s) each comprises a spacer complementary to a genomic sequence within or near (for example, within any of 1, 2, 3, 4, 5, 6, 7, 8, 9, 10, 15, 20, 25, 30, 35, 40, 45, 50, 55, 60, 65, 70, 75, 80, 85, 90, 95, 100 or more bases from) exon 3 of the LPA gene. The gRNA can comprise a spacer sequence complementary to a target sequence within exon 3 of the LPA gene. In some embodiments, a gRNA used in the methods herein comprises a spacer sequence of any one of SEQ ID NOs: 18-25. In some embodiments, a gRNA comprises a spacer sequence of SEQ ID NO: 18 or a sequence having at least 85% homology to the sequence of SEQ ID NO: 18. In some embodiments, a gRNA comprises a sequence of SEQ ID NO: 11 or a sequence having at least 85% homology to the sequence of SEQ ID NO: 11.

In some embodiments, the RNA-guided endonuclease is selected from Casl, CaslB, Cas2, Cas3, Cas4, Cas5, Cas6, Cas7, Cas8, Cas9 (also known as Csn1 and Csxl2), CaslOO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, and Cpf1 endonuclease, or a functional derivative thereof. In some embodiments, the DNA endonuclease is Cas9. In some embodiments, the Cas9 endonuclease is from Streptococcus pyogenes (SpyCas9). In some embodiments, the Cas9 endonuclease is from Staphylococcus lugdunensis (SluCas9). In some embodiments, a DNA sequence that is transcribed to the nucleic acid encoding the DNA endonuclease is codon optimized. In some embodiments, the nucleic acid encoding the DNA endonuclease comprises a 5′ CAP structure and 3′ poly(A) tail. In some embodiments, the nucleic acid encoding the DNA endonuclease is linked to the gRNA via a covalent bond.

In some embodiments, the one or more of the nucleic acid sequences and/or polypeptides can be delivered to cells, either in vitro or in vivo, via viral based or non-viral based delivery systems, including adenovirus vectors, adeno-associated virus (AAV) vectors, retrovirus vectors, lentiviral vectors, herpes virus vectors, nanoparticles, liposomes, lipid nanoparticles, poxviruses, naked DNA administration, plasmids, cosmids, phages, encapsulated cell technology, and the like.

In some embodiments, the compounds of the composition disclosed herein (e.g., the LPA gRNA and the nucleic acid encoding a RNA-guided endonuclease) can be formulated in a liposome or lipid nanoparticle. In some embodiments, the compounds of the composition are formulated in a lipid nanoparticle (LNP). The term “lipid nanoparticle” refers to a nanoscopic particle composed of lipids having a size measured in nanometers (e.g., 1-5,000 nm). In some embodiments, the lipids comprised in the lipid nanoparticles comprise cationic lipids and/or ionizable lipids. Any suitable cationic lipids and/or ionizable lipids known in the art can be used to formulate LNPs for delivery of gRNA and Cas endonuclease to the cells. Exemplary cationic lipids include one or more amine group(s) bearing positive charge. In some embodiments, the cationic lipids are ionizable such that they can exist in a positively charged or neutral from depending on pH. In some embodiments, the cationic lipid of the lipid nanoparticle comprises a protonatable tertiary amine head group that shows positive charge at low pH. The lipid nanoparticles can further comprise one or more neutral lipids, charged lipids, steroids, and polymers conjugated lipids.

In some embodiments, the lipid nanoparticles can have a mean diameter of about, at least, at least about, at most or at most about 30 nm, 40 nm, 50 nm, 60 nm, 70 nm, 80 nm, 90 nm, 100 nm, 110 nm, 120 nm, 130 nm, 140 nm, 150 nm, or a number or a range between any of these values. In some embodiments, the lipid nanoparticle particle size is about 50 to about 100 nm in diameter, or about 70 to about 90 nm in diameter, or about 55 to about 95 nm in diameter. In some embodiments, the plurality of nanoparticles is administered to the subject at a dose of about 0.1-5 mg/kg (determined by the total nucleic acids (e.g., the total of LPA gRNA and Cas9 mRNA)) per administration, including 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, or 5 mg/kg, or a number or a range between any two of these values. In some embodiments, the plurality of nanoparticles is administered to the subject at a dose of, or a dose about, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 2 mg/kg, 2.5 mg/kg or 3.0 mg/kg (in some embodiments, determined by the total of LPA gRNA and SpCas9 mRNA).

In some embodiments, the compounds of the composition described herein are encapsulated in the lipid portion of the lipid nanoparticle or an aqueous space enveloped by some or all of the lipid portion of the lipid nanoparticle. The encapsulation can be full encapsulation, partial encapsulation, or both. In some embodiments, the nucleic acid and/or polypeptides are fully encapsulated in the lipid nanoparticle.

In some embodiments, one or more compounds herein described are associated with a liposome or lipid nanoparticle via a covalent bond or non-covalent bond. In some embodiments, any of the compounds in the composition can be separately or together contained in a liposome or lipid nanoparticle.

In some embodiments, a composition described above can further have one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, any components of a composition are formulated with pharmaceutically acceptable excipients such as carriers, solvents, stabilizers, adjuvants, diluents, etc., depending upon the particular mode of administration and dosage form. In embodiments, guide RNA compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5.0 to about pH 8.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

In some embodiments, the compounds herein described (e.g., a RNA-guided endonuclease or a nucleic acid encoding the RNA-guided endonuclease and/or gRNA) of a composition can be delivered via transfection such as calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the composition is introduced to the cells via lipid-mediated transfection using a lipid nanoparticle.

The composition herein described can be administered to a subject in need thereof to treat a cardiovascular disease or to reduce the risk of developing a cardiovascular disease. Accordingly, the present disclosure also provides a gene therapy approach for treating a cardiovascular disease in a patient or reducing the risk of developing a cardiovascular disease in a subject by editing the LPA gene of the subject. In some embodiments, the gene therapy approach functionally knocks out an LPA gene in the genome of a relevant cell type in patients. The LPA gene of relevant cells in the subject (e.g., hepatocytes) is edited using the materials and methods described herein which used RNA-guided endonuclease, such as Cas9, to permanently delete, insert, edit, correct or replace a target sequence from a genome or insert an exogenous sequence, thereby functionally knocking out the LPA gene. This can provide a permanent cure for the cardiovascular disease by permanently reducing the levels of Lp(a) in the blood.

In some embodiment, a method for treating a cardiovascular disease or disorder in a subject (e.g., a primate subject) in need thereof is disclosed. The method can comprise administering to the primate subject a plurality of nanoparticles complexed with (a) a guide RNA (gRNA) or a nucleic acid encoding a gRNA that targets LPA gene, and (b) a nucleic acid encoding a RNA-guided endonuclease, thereby relieve the cardiovascular disease or disorder in the primate subject. The subject can be administered with the plurality of nanoparticles two or more times, for example twice, for the treatment. Two administration of the nanoparticles to the subject can be separated by a suitable time period. In some embodiments, the suitable time period is, or is about, one week, two weeks, three weeks, four weeks, five weeks, six weeks, seven weeks, eight weeks, three months, four months, five months, six months, a year, two years, three years, or more. In some embodiments, two of the two or more administrations are about two weeks to about two months apart, for example about three weeks. In some embodiments, each two of the two or more administrations are about two weeks to about two months apart, for example about three weeks. In some embodiments, two of the two or more administrations are about one month to about four months apart, for example about two months or three months, or longer. In some embodiments, each two of the two or more administrations are about one month to about four months apart, for example about two months or three months. In some embodiments, two of the two or more administrations are at least two months or three months apart. In some embodiments, each two of the two or more administrations are at least two months or three months apart. In some embodiments, the Lp(a) level in the subject receiving a single administration of the composition herein described can be substantially reduced (e.g., by at least 20%, 30%, 40%, 50%, 70%, 80%, 90%, 91%, 92%, 93%, 94%, 95%, 96%, 97%, 98%, 99% or higher) and remains at the reduced level for at least two months, three months, four months, six months, ten months, one year, eighteen months, two years, three years, four years, five years, ten years, fifteen years, twenty years, or longer after the administration. The suitable time period between two administrations can be the same as or different from the suitable time period between another two administrations. In some embodiments, the plurality of nanoparticles is administered to the subject at a dose of about 0.1-5 mg/kg total RNA (for example, LPA gRNA and Cas9 mRNA), for example 0.5-3 mg/kg total RNA, per administration. In some embodiments, the LPA gRNA or the nucleic acid encoding the LPA gRNA is administered to the subject at a dose of, or a dose of about, 0.1-5 mg/kg, for example 0.1 mg/kg to 5 mg/kg gRNA per administration. In some embodiments, the nucleic acid encoding the RNA-guided endonuclease is administered to the subject at a dose of, or a dose of about, 0.1-5 mg/kg gRNA, for example 0.5-3 mg/kg gRNA per administration. The dose can be the same or different for each of the administration to the subject. In some embodiments, the plurality of nanoparticles is administered to the subject at a dose of about 0.02 to about 1 mg/mL total RNA (e.g., the total of LPA gRNA and Cas9 mRNA), for example about 0.1-0.6 mg/mL total RNA, per administration. The method disclosed herein, in some embodiments, comprises a single administration of the plurality of nanoparticles to the subject. For example, the plurality of nanoparticles can be administered to the subject, in the single administration, at a dose of about 0.5 mg/kg, 0.6 mg/kg, 1 mg/kg, 1.5 mg/kg, 2.0 mg/kg, 2.5 mg/kg, or 3.0 mg/kg of RNA content of (a) the LPA gRNA or the nucleic acid encoding a LPA gRNA, and (b) the nucleic acid encoding a RNA-guided endonuclease. As described herein, a single administration treatment can be effective to the subject, and thus the subject does not require any additional subsequent treatment. For example, in some embodiments, the subject receives a single administration of the plurality of nanoparticles once in a period of three months, four months, five months, six months, one year, two years, three years, four years, five years, six years, seven years, eight years, nine years, ten years, fifteen years, twenty years, twenty-five years, thirty years, thirty-five years, forty years, fifty years, sixty years, or more, or a number or a range between any two of these values.

In some embodiments, the target tissue for the compositions and methods described herein is liver tissue. In some embodiments, the target cells for the compositions and methods described herein is hepatocyte.

In some embodiments, the pharmaceutical composition thereof can be administered by aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, and/or intradermal injection, or any combination thereof. The administration can be local or systemic. The systemic administration includes enteral and parenteral administration. In some embodiments, more than one administration can be employed to achieve the desired level of gene expression over a period of various intervals, e.g., daily, weekly, monthly, or yearly.

The pharmaceutical composition thereof can be administered to a subject in need thereof at a pharmaceutically effective amount. The term “pharmaceutically effective amount” as used herein means that the amount of the pharmaceutical composition that will elicit a desired therapeutic effect and/or biological or medical responses of a tissue, system, animal or human. The administration can result in a desired reduction in the expression of the LPA gene such as a desired reduction in the plasma levels of the Lp(a).

As used herein, the term “cardiovascular disease” refers to a disorder of the heart and blood vessels, and includes disorders of the arteries, veins, arterioles, venules, and capillaries. In some embodiments, the cardiovascular disease is stroke, myocardial infarction, atherosclerosis, familial hypercholesterolemia, atherosclerosis, thrombosis, calcific aortic valve disease, coronary artery disease, peripheral arterial disease, cerebrovascular disease, renal artery stenosis, aortic aneurysm, cardiomyopathy, hypertensive heart disease, heart failure, pulmonary heart disease, congenital heart disease, or rheumatic heart disease. In some embodiments, the methods and compositions herein described can be used to treat calcific aortic valve disease, myocardial infarctions, coronary heart disease, atherosclerosis, thrombosis, stroke or a combination thereof.

In some embodiments, the subject has one or more of the cardiovascular disorder symptoms affecting the heart, brain, one or both legs, pelvis, one or both arms, and/or shoulder. Symptoms of a cardiovascular disorder affecting the heart include, but are not limited to, chest pain, chest discomfort, and pain in one or both arms, one or both shoulders, neck, jaw, or back, shortness of breath, dizziness, faster heartbeats, nausea, abnormal heartbeats, fatigue, and/or myocardial infarction. Symptoms of a cardiovascular disorder affecting the brain include, but are not limited to, sudden numbness or weakness of the face, one or both arms, or one or both legs, sudden confusion or trouble speaking or understanding speech, sudden trouble seeing in one or both eyes, sudden dizziness, difficulty walking, or loss of balance or coordination, and/or sudden severe headache with no known cause. Symptoms of a cardiovascular disorder affecting one or both legs, pelvis, one or both arms, and/or shoulder include, but are not limited to, muscle pain, muscle cramp, cold sensation in one or both feet and/or toes, one or both hands and/or fingers, and/or numbness or weakness in one or both feet and/or toes, one or both hands and/or fingers.

In some embodiments, the subject in need of the treatment can have abnormal levels (higher or lower than the levels in a healthy individual) of total cholesterol, triglycerides, high-density lipoprotein, low-density lipoprotein, complete blood count with differential, Lp(a), Apoliprotein B, homocysteine, hemoglobin A1c, fasting glucose, insulin, creatine kinase, alanine amino-transferase, aspartate trans-aminase, fibrogen, thyroid stimulating hormone, ultra-sensitive C-reactive protein, urine albumin creatinine ratio, MPO, vitamin D, trimethylamine N-oxide, aminoterminal, pro-brain natriuretic peptide, serum creatinine, global risk score, or a combination thereof.

In some embodiments, the subject may have been pretreated with steroids and/or antihistamine drugs. For example, the subject may have been pretreated with dexamethasone, famotidine, diphenhydramine, or a combination thereof. The pretreatment can be performed about, at least, at least about, at most or at most about 30 minutes, 1 hour, 2 hours, 4 hours, 8 hours, 16 hours, 1 day, 2 days, 5 days, 10 days prior to the administration.

In some embodiments, the method further comprises identifying a subject in need of the treatment. In some embodiments, the method further comprises measuring the blood level of Lp(a) in the primate subject prior to, during, and/or after the administration.

In some embodiments, the subject has high levels of Lp(a) (e.g., plasma Lp(a)) prior to the administration. A subject with high levels of Lp(a) can include, e.g., subjects with greater Lp(a) levels than 90% of the human population. In some embodiments, the subject has symptoms of a cardiovascular disease. In some embodiments, the subject does not have symptoms of a cardiovascular disease. In some embodiments, the subject is at risk of developing a cardiovascular disease. In some embodiments, the subject is suspected of having or developing a cardiovascular disease.

In some embodiments, the subject prior to the administration has plasma Lp(a) levels in excess of about 30 mg/dL (such as in excess of about any of 40 mg/dL, 50 mg/dL, 60 mg/dL, 70 mg/dL, 80 mg/dL, 100 mg/dL, 125 mg/dL, 150 mg/dL, 175 mg/dL, 200 mg/dL, 225 mg/dL, 250 mg/dL, 275 mg/dL, 300 mg/dL, or greater). In some embodiments, the subject in need of the treatment has a plasma Lp(a) level greater than 50 mg/dL. In some embodiments, the subject prior to the administration has plasma Lp(a) levels in excess of about 30 mg/dL (such as in excess of about any of 100 nmol/L, 125 nmol/L, 150 nmol/L, 175 nmol/L, 200 nmol/L, 225 nmol/L, 250 nmol/L, 275 nmol/L, 300 nmol/L, 350 nmol/L, 400 nmol/L, or greater). In some embodiments, the subject has one or more genetic markers (e.g., deletion, insertion, and/or mutation) in the endogenous LPA gene or its regulatory sequences such that the activity, including the expression level or functionality, of the apo(a) protein is substantially increased compared to a normal, healthy subject.

In some embodiments, the subject in need of the treatment is a patient having high levels of Lp(a) as defined as Lp(a) levels higher than 90% of the human population (e.g., higher than 60 mg/dL) and symptoms of a lipoprotein-related disease (e.g., a cardiovascular disease). In some embodiments, the subject can be a human suspected of having the lipoprotein-related disease. Alternatively, the subject can be a human diagnosed with a risk of the lipoprotein-related disease due to the presence of Lp(a) levels in excess of 60 mg/dL or 70 mg/dL or 80 mg/dL or 100 mg/dL or 200 mg/dL or 300 mg/dL. In some embodiments, the subject in need of the treatment can have a LPA gene variant associated with increased lipoprotein-related disease risk and/or increased Lp(a) expression. In some embodiments, the subject is a mammal. In some embodiments, the subject is a primate. In some embodiments, the subject is a human.

The methods and compositions herein described can reduce the plasma low-density lipoprotein (LDL) levels such as the plasms Lp(a) levels, therefore reducing the risk of cardiovascular disease, such as the risk of heart attack, stroke, blood clots, fatty build-up in veins and other coronary artery disease, the likelihood of mortality related to cardiovascular events, or a combination thereof. In some embodiments, the methods and compositions herein described can reduce or relieve one or more symptoms of the cardiovascular disease.

In some embodiments, the plasma Lp(a) level in the subject following carrying out the method is reduced to about 50 mg/dL or lower (e.g., about, at most, or at most about 40 mg/dL, 30 mg/dL, 20 mg/dL, or lower). In some embodiments, the plasma Lp(a) level in the subject following carrying out the method is reduced to about 40 mg/dL or lower. In some embodiments, the plasma Lp(a) level in the subject following carrying out the method is reduced to about 30 mg/dL or lower. In some embodiments, the plasma Lp(a) level in the subject following carrying out the method is reduced to about 20 mg/dL or lower.

In some embodiments, the plasma Lp(a) level in the subject following carrying out the method is reduced by about, at least, or at least about 50%, 60%, 70%, 80%, 90%, 95%, 98%, 99%, or a number or a range between any of these values.

In some embodiments, the plasma LDL level in the subject following carrying out the method is reduced by about, at least, or at least about 25%, 30%, 40%, 50%, 60%, 70%, 80%, 90%, 95%, 98%, 99%, or a number or a range between any of these values.

Provided herein also includes kits for carrying out the methods described herein. A kit can include a genome-targeting nucleic acid (e.g., gRNA targeting the LPA gene) and a RNA-guided endonuclease (e.g., Cas9) or a nucleic acid encoding the RNA-guided endonuclease. In any of the above kits, the kit can further comprise a polynucleotide to be inserted to effect the desired genetic modification (e.g., a donor template). Components of a kit can be in separate containers, or combined in a single container.

Any kit described above can further comprise one or more additional reagents selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the endonuclease, or improve the specificity of targeting.

In some embodiments, a kit can further include instructions for using the components of the kit to practice the methods described herein. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g., CD-ROM, diskette, flash drive, etc. In some instances, the actual instructions are not present in the kit, but means for obtaining the instructions from a remote source (e.g., via the Internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

Some aspects of the embodiments discussed above are disclosed in further detail in the following examples, which are not in any way intended to limit the scope of the present disclosure.

### Example 1

In Vitro Validation of LPA-Targeted gRNA Spacers

To evaluate the ability of gRNAs to affect targeted cleavage. Space sequences described herein in Table 1 were synthesized by in vitro transcription (IVT gRNA) and evaluated in a system using transfection into a human embryonic kidney (HEK) cell line engineered to constitutively express the SpCas9 nuclease. The cleavage efficiency at the on-target site for each gRNA was measured using the TIDES protocol (Brinkman, E. K. et al. (2014). Nucleic Acids Research, 42(22):e168), in which PCR primers flanking the predicted cleavage site are used to amplify the genomic DNA from treated cells, followed by Sanger sequencing of the PCR product. When a double-strand break is created in the genome of a cell, the cell attempts to repair the double-strand break. This repair process is error prone, which can result in the deletion or insertion of nucleotides at the site of the double-strand break. Because breaks that are perfectly repaired are re-cleaved by the Cas9 nuclease, whereas insertion or deletion of nucleotides will prevent Cas9 cleavage, there will be an accumulation of insertions and deletions that are representative of the cutting efficiency. The sequencing chromatogram data were then analyzed using a computer algorithm that calculates the frequency of inserted or deleted bases at the predicted cleavage site. The frequency of inserted or deleted bases (INDELs) was used to calculate the overall cleavage frequency. However, due to the repetitive nature of some of the kringle IV regions, PCR amplification was not possible. For these guide RNA targets, INDELs could not be assayed using this method. The results from the IVT gRNA experiment in HEK cells are shown in Table 3, where the gRNAs are ranked according to their observed cutting efficiencies. In Table 3, Y=100% match to Macaca fascicularis.

TABLE 3
Cleavage efficiency of guide RNA molecules
targeting the LPA gene in HEK cells

| Guide | Region of gene | NHP Match | INDEL Frequency (%) |
|---|---|---|---|
| T4 | Exon 3 | Y | 1.7 |
| T1 (T5513) | Exon 3 | Y | 6.1 |
| T2 (T13789) | Exon 3 | Y | 2.4 |
| T10762 | Exon 3 | — | 1.9 |
| T3893 | Exon 3 | — | 2.2 |
| T3894 | Exon 3 | — | 1.9 |
| T14467 | Exon 3 | — | 1.5 |
| T5 (T1530) | Exon 3 | Y | 0.9 |

### Example 2

### Evaluation of LPA Gene Editing Efficiency in Non-Human Primates (NHIPs)

This example evaluates the LPA gene editing efficiency in NHPs by measuring the plasma Lp(a) levels before and after treatments.

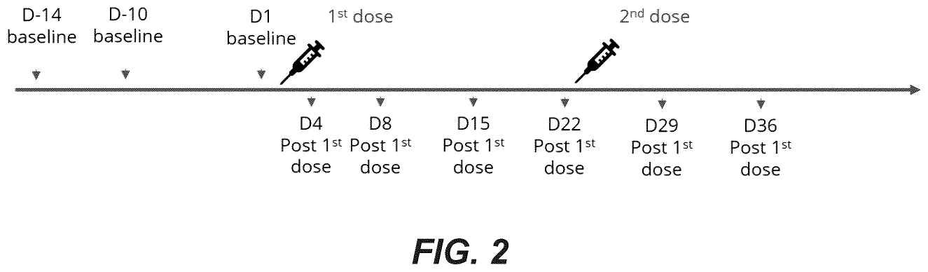

NHPs were pre-screened for SNPs in the LPA gene and the Lp(a) plasma baselevels were measured. T4 gRNA (SEQ ID NO: 3) was selected to match the LPA gene of the NHPs. A lipid nanoparticle (LNP) delivery vehicle was used to deliver Cas9 mRNA and T4 gRNA molecules to the NHPs. LNPs encapsulating the gRNA molecule and Cas9 mRNA were rejected into the NHP according to the study design shown in FIG. 2. In particular, each NHP was injected with 2 doses of a RNA097 Cas9 mRNA (Pseudouridine modified) and T4 gRNA encapsulated in Gen3 LNP (Group 4) or one dose of a RNA010 Cas9 mRNA (N-Methyl Pseudouridine modified) and T4 gRNA encapsulated in Gen3 LNP (Group 5). The plasma samples were collected and plasma Lp(a) levels were monitored before and after treatment. A Sandwich Enzyme-Linked Immunosorbent Assay (ELISA) was used to detect concentrations of Lp(a) protein in the plasma collected. Efficient editing was observed, including about 50% liver editing at 2 mg/kg dose.

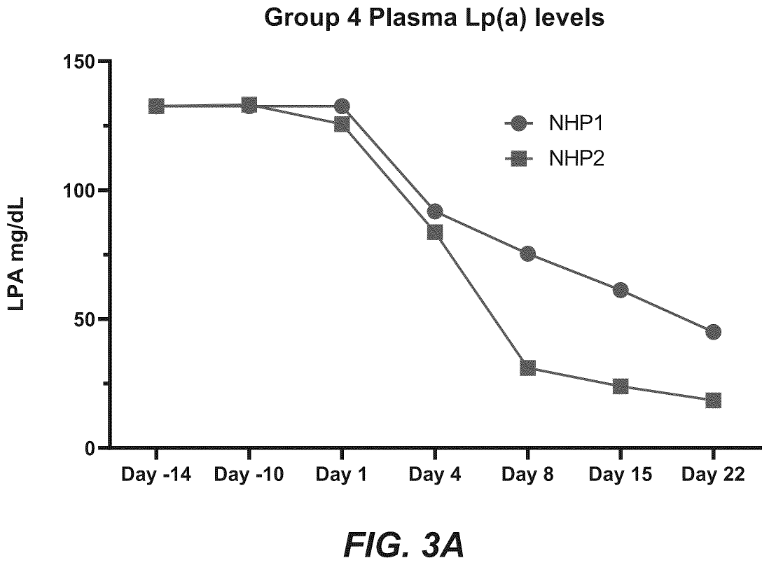

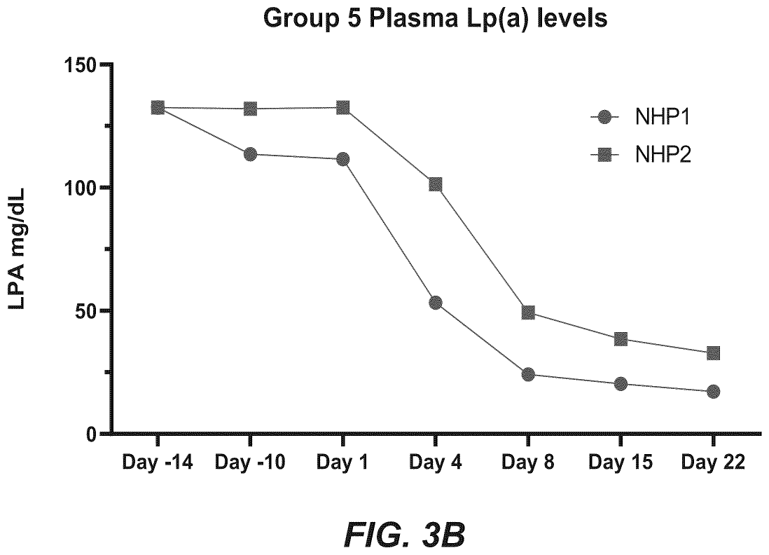

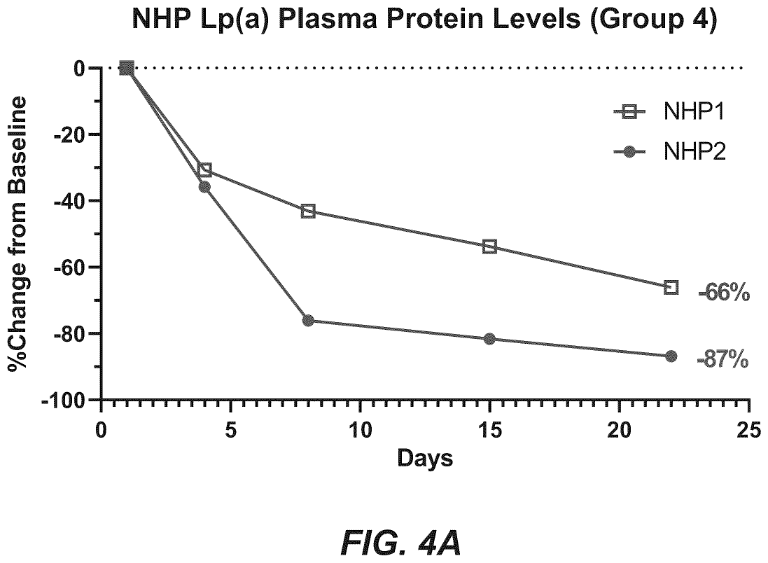

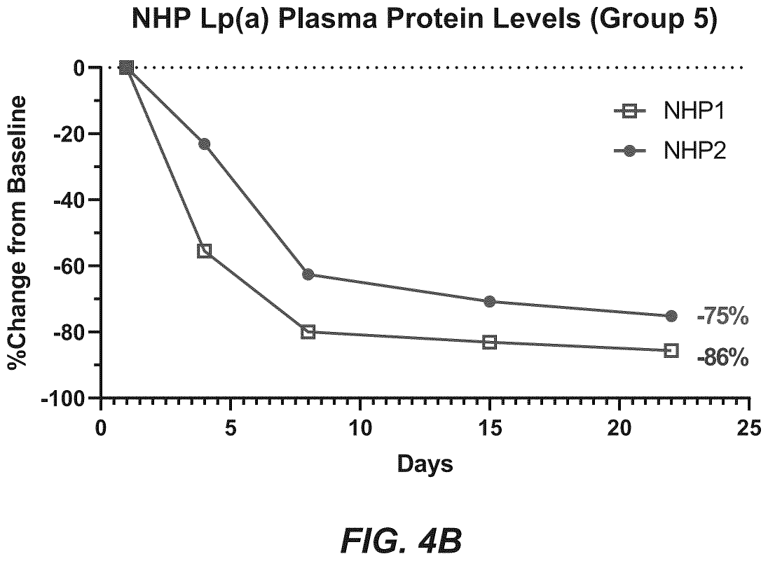

FIG. 3A-B are two graphs showing the plasma Lp(a) levels of Group 4 NHPs (FIG. 3A) and Group 5 NHPs (FIG. 3B). FIG. 4A-B are two graphs showing the percentage change of the plasma Lp(a) from baseline of Group 4 NHPs (FIG. 4A) and Group 5 NHPs (FIG. 4B).

The results demonstrate that the NHP plasma Lp(a) levels are significantly decreased using the T4 gRNA and Cas9 mRNA. 66% and 87% decrease from baseline were observed in Group 4 and 75% and 86% observed in Group 5.

### Example 3

Toxicity Study of a LPA gRNA Formulation

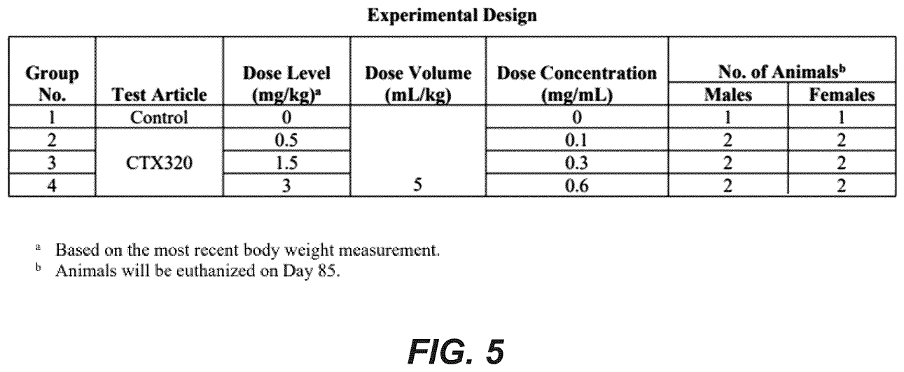

In this example, a toxicity study was carried out in NHPs. Lipid nanoparticles encapsulating the gRNA molecule of SEQ ID NO: 3 (T4 gRNA) and Cas9 mRNA were formulated to a LPA gRNA formulation referred to as “CTX320”. The CTX320 formulation was administered in a single dose into three groups of NHPs (e.g., cynomolgus monkey) at a dose level of 0.5 mg/kg, 1.5 mg/kg, and 3.0 mg/kg, respectively. Plasma Lp(a) protein level was measured using Mercodia ELISA assay. FIG. 5 depicts a non-limiting exemplary experimental design.

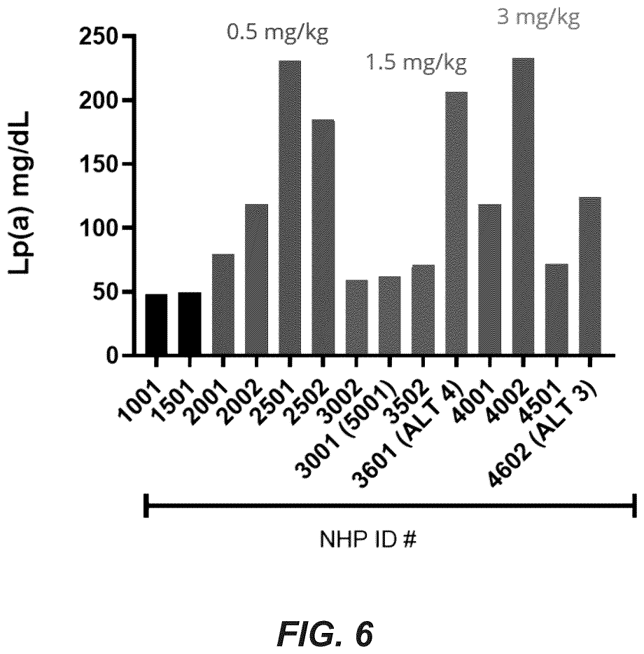

FIG. 6 is a graph showing the baseline Lp(a) levels in NHP plasma fourteen days prior to the treatment (Day −14). Baseline values vary between individual NHPs due to the number of Kringle IV-2 repeats.

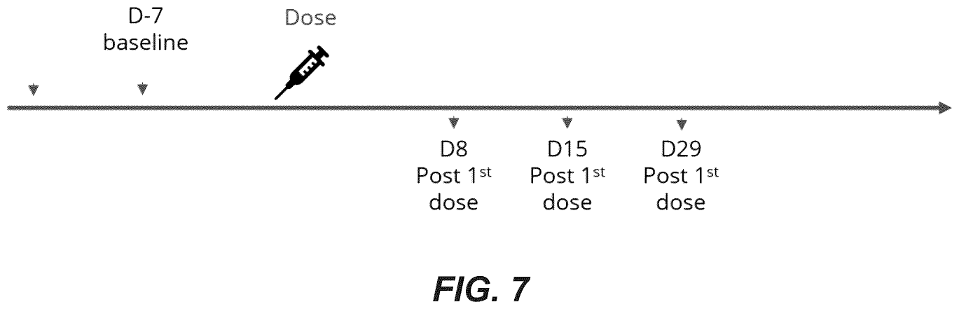

FIG. 7 depicts a non-limiting exemplary NHP study design. Plasma Lp(a) protein levels were measured at 7 days prior to the treatment and 8 days, 15 days and 29 days after the treatment.

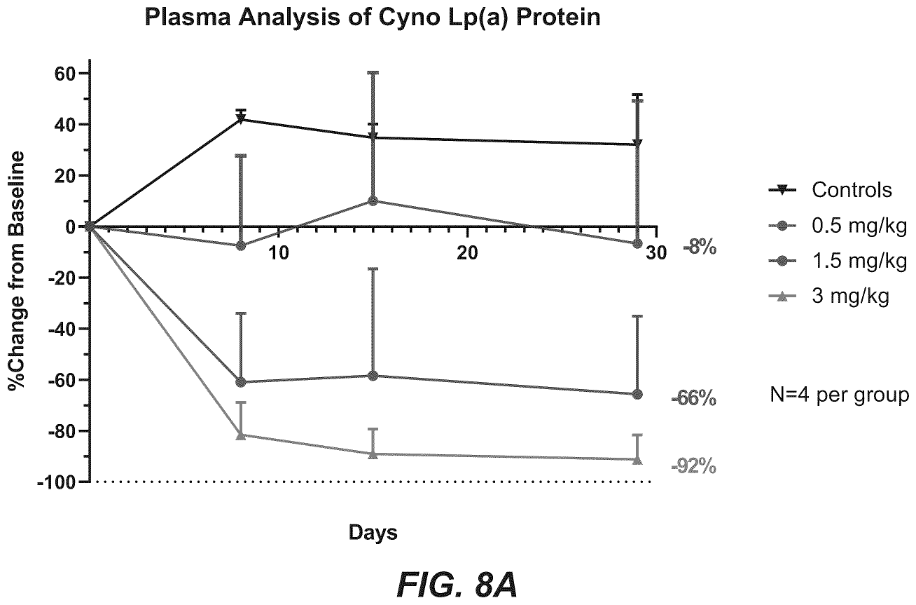

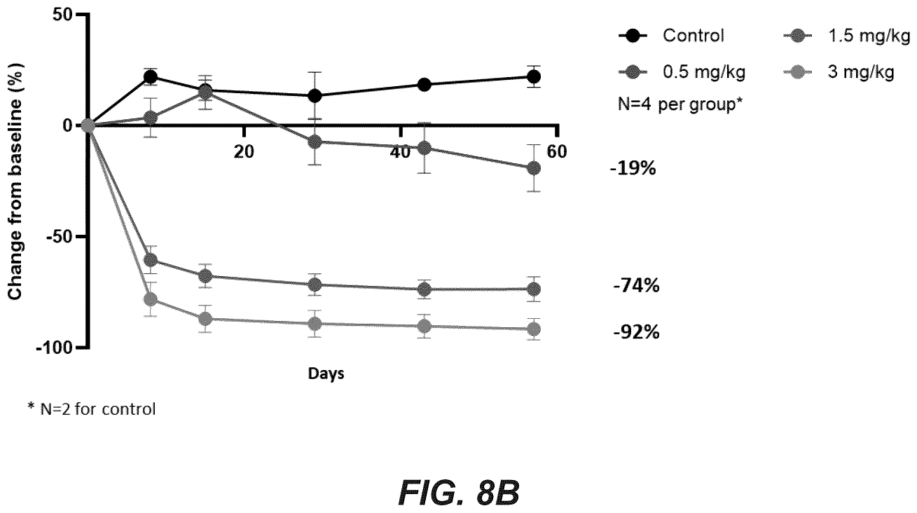

FIG. 8A is a graph showing the percentage change of the plasma Lp(a) protein levels from a baseline of NHPs after the CTX320 treatments with three different doses: 0.5 mg/kg, 1.5 mg/kg, and 3 mg/kg in comparison to a control group. FIG. 8B is a graph showing the percentage change of the serum Lp(a) protein levels from a baseline of NHPs after the CTX320 treatments with three different doses: 0.5 mg/kg, 1.5 mg/kg, and 3 mg/kg in comparison to a control group. The data demonstrates a dose-dependent reduction of plasma and serum Lp(a) protein after CTX320 dosing. About 66% reduction of Lp(a) protein from baseline was observed in NHPs treated with 1.5 mg/kg CTX320, and about 92% reduction in NHPs treated with 3.0 mg/kg CTX320. No significant reduction of plasma Lp(a) protein was observed with the 0.5 mg/kg CTX320 dosing.

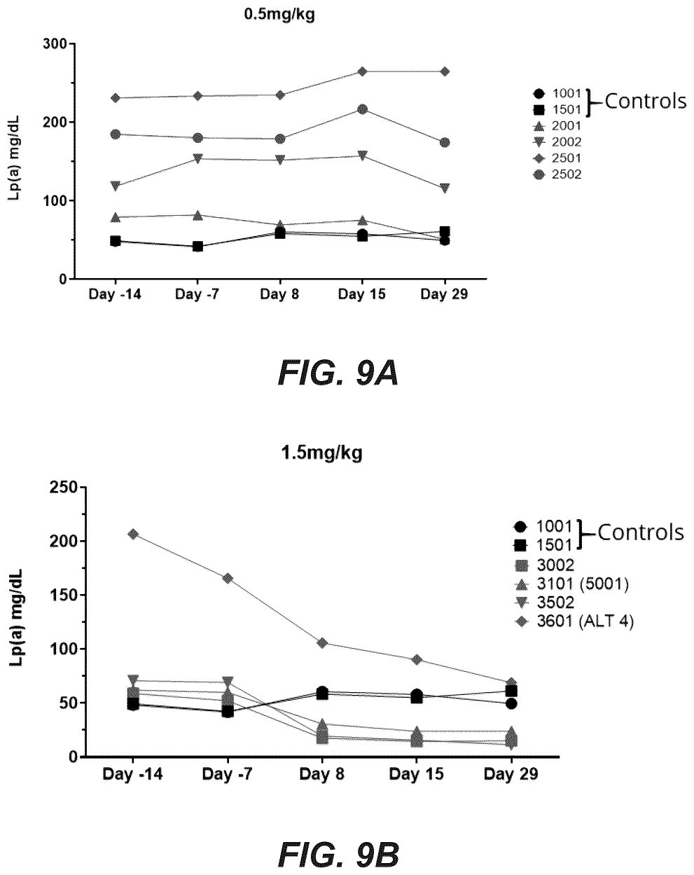

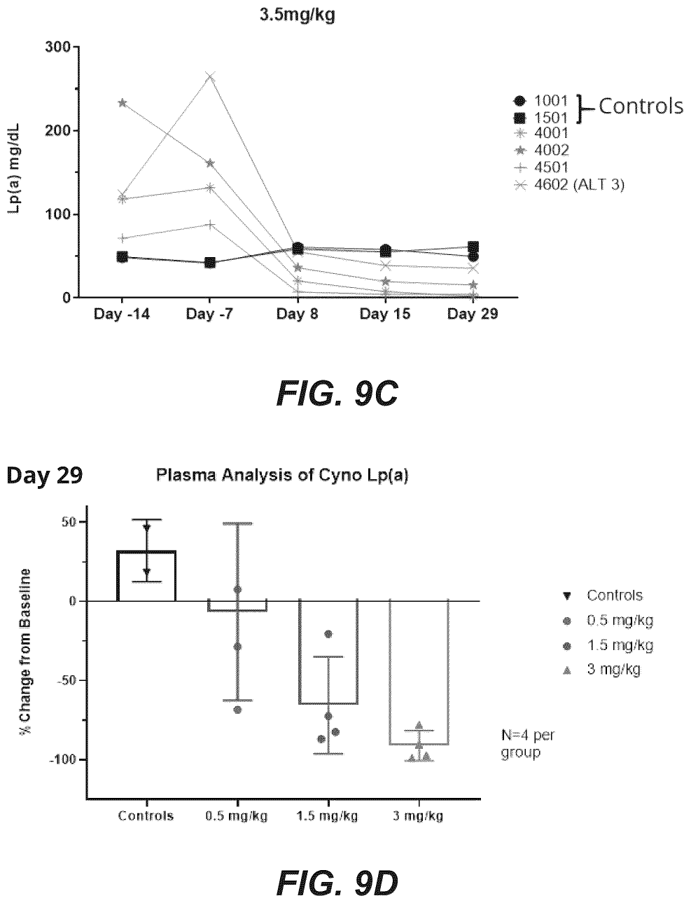

FIGS. 9A-C are graphs showing the plasma Lp(a) protein levels of the NHPs before and after treatments with three different doses of CTX320: 0.5 mg/kg (FIG. 9A), 1.5 mg/kg (FIG. 9B) and 3.0 mg/kg (FIG. 9C). FIG. 9D shows the percentage change of the plasma Lp(a) level from the baseline of the NHPs on Day 29 after the CTX320 treatments. An average of 50% or greater reduction of the plasma Lp(a) levels from baseline was observed in NHPs treated with 1.5 mg/kg and 3 mg/kg CTX320 formulation.

Serum tests were also carried out for the NHPs following the CTX320 treatment, including liver function tests, plasma levels of lipids (e.g., triglyceride, HDL, LDL and total cholesterol), kidney function tests (e.g., blood urea nitrogen, creatinine, glucose, and calcium), markers of injury (e.g., lactate dehydrogenase (LDH), gamma glutamyl transferase, and c-reactive protein (CRP)), leukocytes, electrolyte panel, red cell indices, coagulation profile, and urine tests. The data (not shown) demonstrates that transient dose-dependent increase in liver enzymes and total bilirubin reached a peak at about 2-4 days following the treatments and returned to baseline by Day 15. Transient increase in LDH and CRP peaked at Day 2 and returned to baseline by Day 7. The body weight of the NHPs also remain stable following the CTX320 treatments. The data also suggests that 3 mg/kg CTX320 formulation did not alter cholesterol levels and induced an transient increase in triglyceride and LDL levels that peaked at Days 2-4 following the treatment, as well as a sharp decline in HDL levels at Day 4 following the treatment. Furthermore, it was found that treatment with CTX320 did not alter functions of markers of kidney (including blood urea nitrogen and creatinine).

### Example 4

Efficacy Assessment and Toxicity Study of a LPA gRNA Formulation

This example reports another non-limiting exemplary efficacy and toxicity assessment of the CTX320 formulation of Example 3.

The CTX320 formulation was administered in a single dose into three groups of NHPs (e.g., cynomolgus monkey) at a dose level of 0.5 mg/kg, 1.5 mg/kg, and 3.0 mg/kg, respectively, according to the experimental design of FIG. 5. Plasma Lp(a) protein level was measured throughout the study using Mercodia ELISA assay. Animals were euthanized on Day 85 following the treatment.

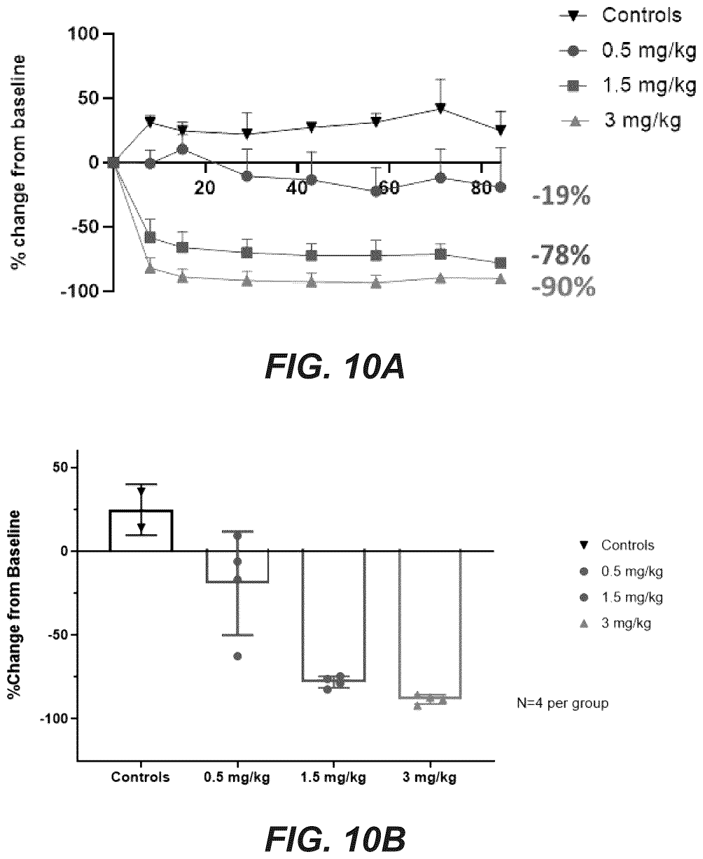

FIG. 10A is a graph showing the percentage change of the serum Lp(a) protein levels from a baseline of NHPs after the CTX320 treatments with three different doses: 0.5 mg/kg, 1.5 mg/kg, and 3 mg/kg in comparison to a control group. The data demonstrates a dose-dependent reduction of plasma and serum Lp(a) protein after CTX320 dosing. About 78% reduction of Lp(a) protein from baseline was observed in NHPs treated with 1.5 mg/kg CTX320, and about 90% reduction in NHPs treated with 3.0 mg/kg CTX320. About 19% reduction of plasma Lp(a) protein was observed with the 0.5 mg/kg CTX320 dosing. As shown in FIG. 10A, a single dose of CTX320 results in sustained reduction of Lp(a) and the significant reduction in Lp(a) levels were sustained during the study duration at the intermediate and high doses.

FIG. 10B shows the percentage change of the plasma Lp(a) level from the baseline of the NHPs after about 3-month CTX320 treatment. Significant reduction in Lp(a) levels from baseline were observed in NHPs treated with 1.5 mg/kg and 3 mg/kg CTX320 formulation. The results demonstrate that a single dose of CTX320 can result in sustained reduction of Lp(a) from baseline even after 3-month following the treatment at the intermediate and high doses.

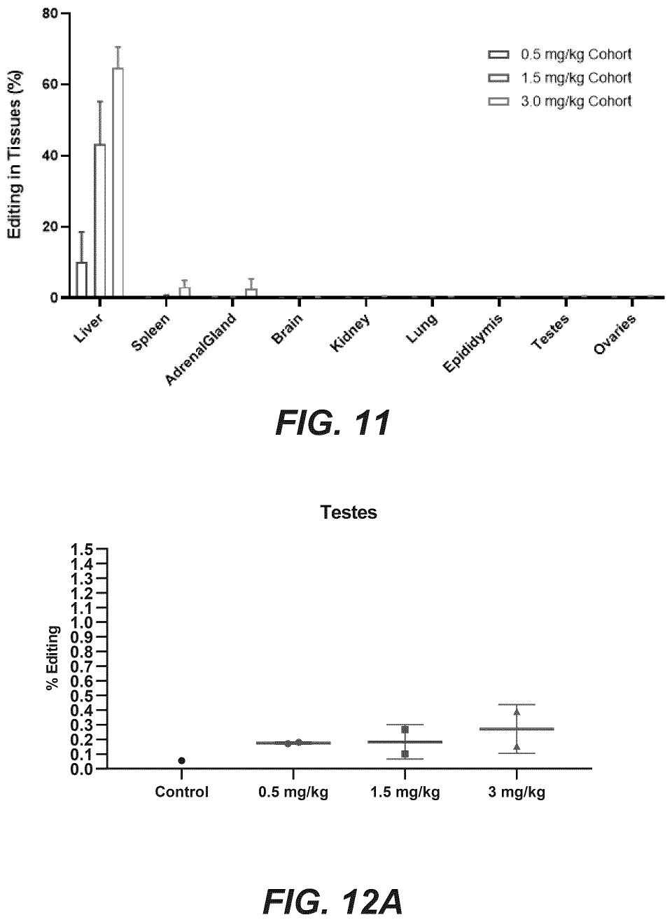

FIG. 11 is a plot showing the percentage of LPA gene editing in liver and other organ tissues including spleen, adrenal gland, brain, kidney, lung, epididymis, testes and ovaries. The results demonstrate that liver is the predominant organ of LPA gene editing, while only about 0.1-5% LPA gene editing was observed in spleen after about 3-month CTX320 treatment. Additionally, dose dependent editing was also observed in liver 85 days post treatment. About 40% LPA and 65% gene editing was observed following treatment with 1.5 mg/kg CTX320 and 3 mg/kg CTX320, respectively. Some results of LPA on-target editing in tissues are shown in Table 4.

TABLE 4
3 month (% indel) data for LPA on-target editing in tissues

| | Control | | 0.5 mg/kg | | 1.5 mg/kg | | 3.0 mg/kg | |
|---|---|---|---|---|---|---|---|---|
| Tissue | Average | SD | Average | SD | Average | SD | Average | SD |
| Liver | 0.22 | 0.15 | 9.96 | 8.55 | 43.34 | 11.87 | 64.80 | 5.77 |
| Spleen | 0.10 | 0.01 | 0.13 | 0.05 | 0.44 | 0.32 | 3.10 | 1.87 |
| Adrenal | 0.25 | 0.29 | 0.20 | 0.11 | 0.14 | 0.12 | 2.72 | 2.68 |
| Gland | | | | | | | | |
| Brain | 0.12 | 0.05 | 0.08 | 0.04 | 0.10 | 0.06 | 0.20 | 0.07 |
| Kidney | 0.14 | 0.00 | 0.10 | 0.07 | 0.10 | 0.03 | 0.30 | 0.12 |
| Lung | 0.12 | 0.04 | 0.13 | 0.06 | 0.22 | 0.12 | 0.24 | 0.11 |
| Epididymis | 0.12 | N/A | 0.08 | 0.01 | 0.10 | 0.02 | 0.15 | 0.19 |
| Testes | 0.06 | N/A | 0.18 | 0.01 | 0.18 | 0.12 | 0.27 | 0.17 |
| Ovary | 0.15 | N/A | 0.12 | 0.09 | 0.14 | 0.01 | 0.23 | 0.20 |

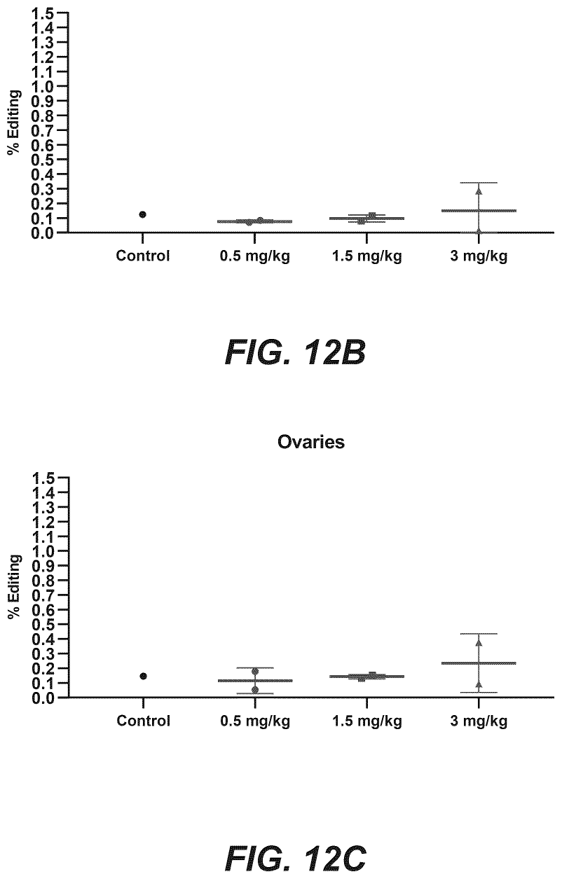

Additional data (FIGS. 12A-12C) also demonstrate a dose-dependent editing in reproductive tissues of testes, epididymis and ovaries, with less than 0.5% editing 85 days post treatment with 3 mg/kg CTX320 and even lower with 0.5 mg/kg and 1.5 mg/kg CTX320.

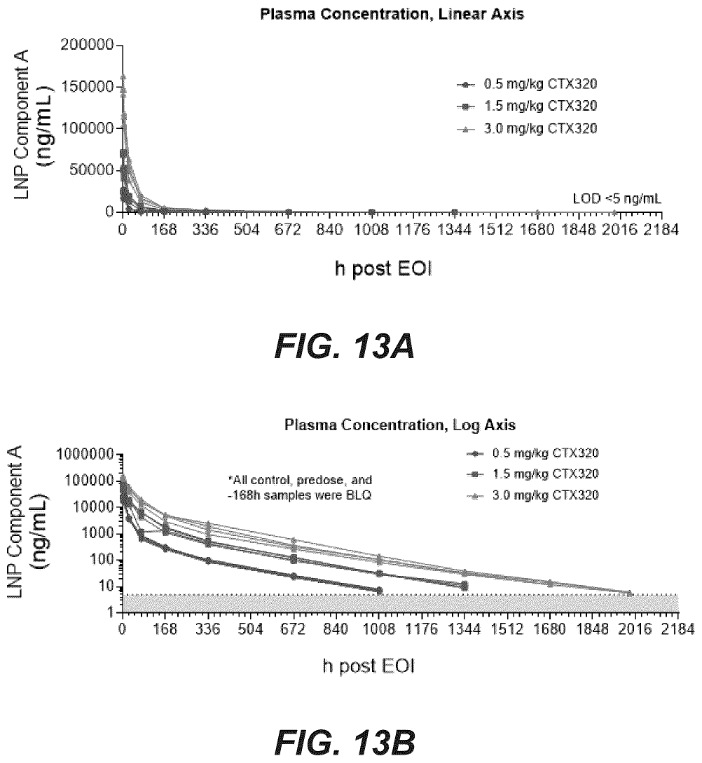

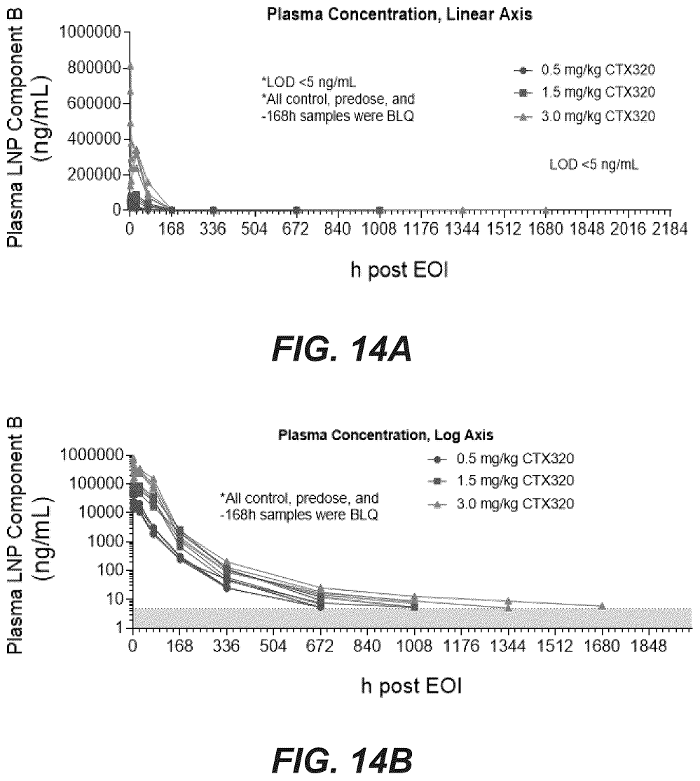

FIGS. 13A-B are plots showing the plasma level of LNP component A post treatment. FIGS. 14A-B are plots showing the plasma level of LNP component B post treatment. The toxicokinetic analysis suggests that most of LNP component A and B are cleared from the plasma one week following treatment with CTX320. Both LNP components A and B are excreted by 168 hours post injection.

The data (not shown) indicates that CTX320 causes transient dose-dependent elevations in liver functional tests (e.g., alanine aminotransferase, aspartate aminotransferase, alkaline phosphatase, and bilirubin). No liver functional impairment or damage was detected after the treatment (e.g., 85 days after the administration).

Additional endpoint analysis including body weight, clinical signs (e.g., aspartate transaminase (AST), alanine aminotransferase (ALT), alkaline phosphatase (ALP), total bilirubin), urinalysis, hematology and coagulation, electrocardiogram and histopathology (both gross- and micro-evaluation) reveals no CTX320 related change throughout the study duration.

### Example 5

### Phase 1 Study Design

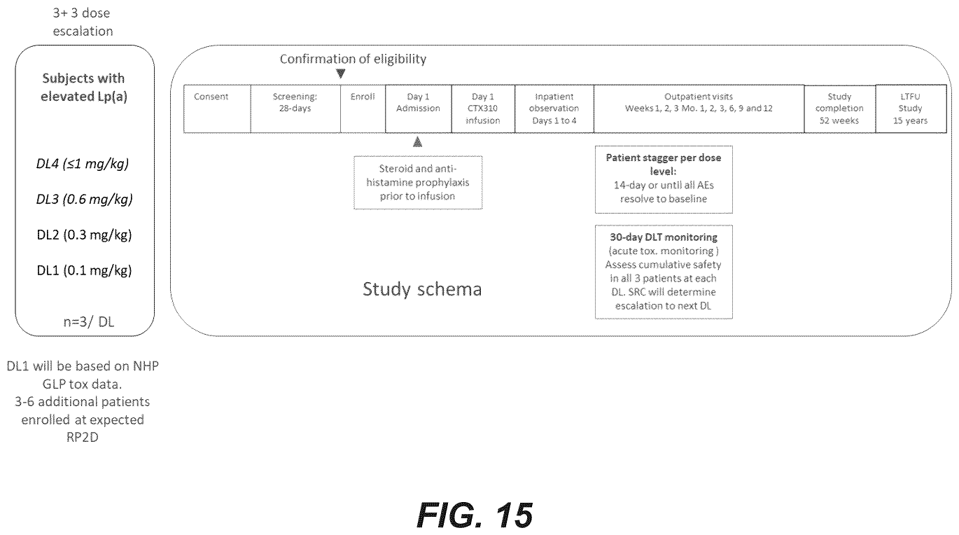

A non-limiting exemplar design for Phase 1 safety and tolerability clinical study for one or more of the LPA gene-editing nanoparticle described herein (e.g., CTX320) is shown in FIG. 15.

In some cases, Phase 1 clinical study of CTX320 is conducted in patients with elevated Lp(a) levels. For example, the patient can have Lp(a)≥50 mg/dL or ≥100 nmol/L with increased risk of myocardial infarction (MI) independent of established CVD risk factors. In some cases, the patient can have Lp(a)≥50 mg/dL or ≥125 nmol/L. In some cases, the patient can have Lp(a) levels>180 mg/dL (>430 nmol/L) with an increased lifetime risk of atherosclerotic cardiovascular disease (ASCVD). The patient can be between the age of 18 and 75. In some cases, HBA1C test is performed about every 3 months for patients.

In some cases, subjects with advanced liver disease are excluded from the study. Advanced liver diseases can include: (a) Aspartate transaminase (AST), alanine transaminase (ALT)>3×the upper limit of normal (ULN), or direct bilirubin value>2×the ULN, and/or (b) Baseline prothrombin time (International Normalized Ratio [INR])>1.5×ULN, and/or (c) Fibroscan or MRE read of ≥7.5 kpa, and/or (d) History of hepatic cirrhosis, and/or (e) History of alcohol or drug abuse, and/or (f) Acute monitoring of ALT, AST, GGT, Bili., Alk. Phos., albumin, INR, PT and PTT through D30. Monitoring will continue through the end of study (12 months).

### Example 6

Durability Study of a LPA gRNA Formulation

This example reports a non-limiting exemplary durability trial study of the CTX320 formulation of Example 3. In particular, this example evaluates the durability of LPA protein knock-down in plasms using ELISA and assesses the LPA disruption in liver tissue. The durability study also assesses whether pre-treatment with steroids and/or anti-histamine reduces liver function test elevations. Table 5 shows a durability study design used in this example.

TABLE 5
An exemplary durability study design

| | Group | Dose | | LPA | |
|---|---|---|---|---|---|
| Formulation | size | Level | LPA ELISA | editing | Endpoints |
| CTX320 with | 4 (2 | 2.0 | 7 d, 14 d, 21 d, | 12 months, | Necropsy: 2 years |
| pre-treatment | Male + 2 | mg/kg | 28 d, 6 wk, 8 wk, | 18 months, | Biopsy: 12 and 18 |
| | Female) | | 10 wk, 12 wk, | 24 months - | months |
| | | | monthly (4 | liver only | Serum Chemistry |
| | | | months-24 | | PD Samples |
| | | | months) | | Bioanalysis/TK Samples |

The patients selected have an elevated LP(a) level greater than 100 mg/dL. The patients can be pretreated with steroids and/or antihistamine drugs. For example, the patients can be pretreated with 1 mg/kg dexamethasone, 0.5 mg/kg famotidine, and 5 mg/kg diphenhydramine on the day before LNP administration and then again 30-60 min before LNP administration.

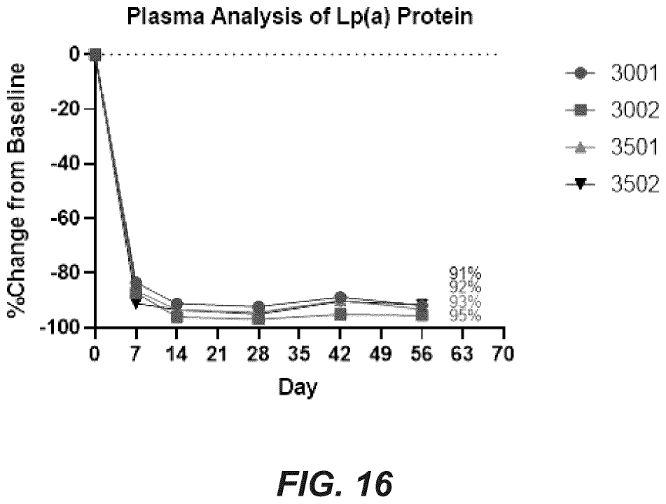

FIG. 16 is a graph showing the percentage change of the serum Lp(a) protein levels from a baseline of patients after CTX320 treatments. 56 days after the administration at least 90% reduction of Lp(a) protein from baseline was still observed in patients treated with CTX320.

Serum tests were also carried out for the patients following the CTX320 treatment, including liver function tests (LFTs) and plasma levels of lipids (e.g., triglyceride, HDL, LDL and total cholesterol).

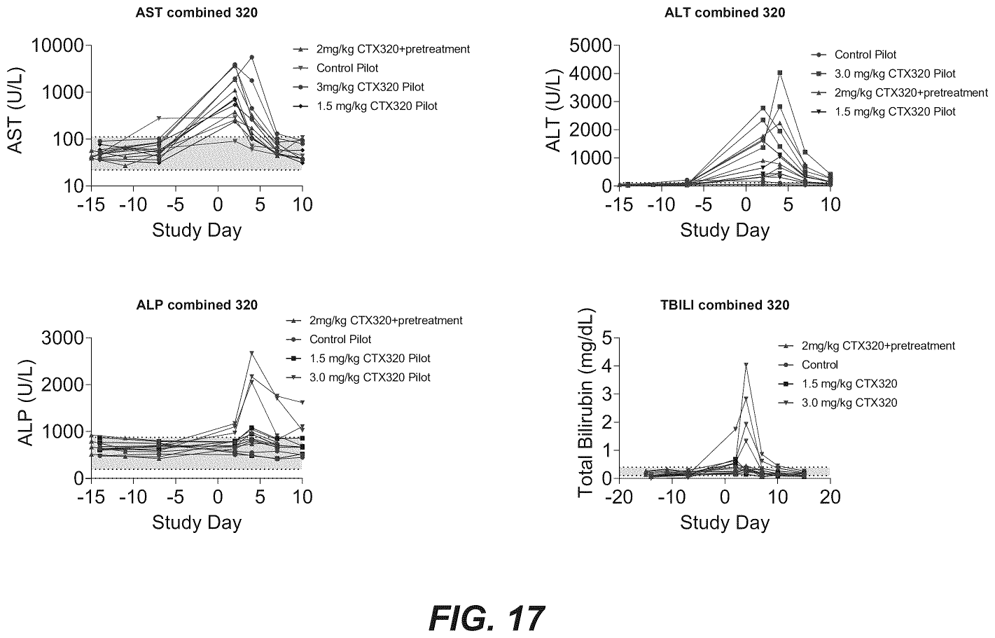

FIG. 17 compares aspartate aminotransferase (AST), alanine transaminase (ALT), alkaline phosphatase (ALP) and total bilirubin levels in patients with pre-treatment and 2.0 mg/kg CTX320 formulation and in patients without pretreatment and with 1.5 mg/kg and 3 mg/kg CTX320 formulation. The data suggests that CTX320 formulation with pretreatment induces LFT elevations similar to those observed in previous studies without pretreatment and that the pretreatment with antihistamine does not dampen LFT responses in patients.

It was also observed that CTX320 also induced decreases in total cholesterol and LDL. No appreciable difference in other serum chemistry markers was found to be induced by CTX320 formulation.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for treating a lipoprotein-related disease in a subject in need thereof, comprising administering to the subject a plurality of nanoparticles complexed with
(a) a guide RNA (gRNA) targeting LPA gene (LPA gRNA) or a nucleic acid encoding a LPA gRNA; and
(b) a nucleic acid encoding a RNA-guided endonuclease,
thereby treating the lipoprotein-related disease in the subject.

2. The method of claim 1, wherein the subject is administered with the plurality of nanoparticles two or more times.

3. The method of any one of claims 1-2, wherein each two of the two or more administrations are about two weeks to about four weeks apart.

4. The method of any one of claims 1-3, wherein each two of the two or more administrations are at least three months apart.

5. The method of claim 1, wherein the method comprises a single administration of the plurality of nanoparticles to the subject.

6. The method of any one of claims 1-5, wherein the LPA expression in the plasma of the subject is reduced by at least 20%, by at least 40%, by at least 70%, or by at least 90% after the administration.

7. The method of any one of claims 1-5, wherein the concentration of LPA protein in the plasma of the subject is reduced by at least 20%, by at least 40%, by at least 70%, or by at least 90% after the administration.

8. The method of any one of claims 5-7, wherein the reduction is for three weeks, four weeks, five weeks, two months, three months or longer after the administration.

9. The method of any one of claims 5-7, wherein the reduction is 65% one month after the administration.

10. The method of any one of claims 5-9, wherein the reduction is relative to (a) the LPA expression or the concentration of LPA protein in the plasma of the subject prior to being administered with the plurality nanoparticles; (b) the LPA expression or the concentration of LPA protein in one or more untreated subjects; and/or (c) a reference level of LPA expression or the concentration of LPA protein of healthy subjects.

11. The method of any one of claims 1-10, wherein the subject in need has a Lp(a) level of more than 50 mg/dl

12. The method of any one of claims 1-10, wherein the subject in need has a Lp(a) level of more than 100 mg/dL.

13. The method of any one of claims 1-12, wherein the subject in need has an increased risk of myocardial infarction (MI) independent of established cardiovascular disease (CVD) risk factors, or an increased lifetime risk of atherosclerotic cardiovascular disease (ASCVD).

14. The method of any one of claims 1-13, comprising measuring the blood or serum level of Lp(a) in the subject prior to, during, and/or after the administration.

15. The method of any one of claims 1-13, comprising identifying a subject in need of the treatment.

16. The method of any one of claims 1-15, wherein the lipoprotein-related disease is a metabolic disease, a cardiovascular disease, a lipid metabolism disease, or a combination thereof.

17. The method of any one of claims 1-15, wherein the lipoprotein-related disease is calcific aortic valve disease, myocardial infarctions, coronary heart disease, atherosclerosis, thrombosis, stroke, coronary artery disease, familial hyperlipidemia, myocardial infarction, peripheral arterial disease, calcific aortic valve stenosis, or a combination thereof.

18. The method of any one of claims 1-17, wherein one or more symptoms of the lipoprotein-related disease in the subject is reduced or relieved.

19. The method of any one of claims 1-18, wherein the administering to the subject a plurality of nanoparticles complexed reduces cardiovascular risk, likelihood of mortality related to cardiovascular events, or a combination thereof.

20. The method of any one of claims 1-19, wherein the nucleic acid encoding a RNA-guided endonuclease is a mRNA of the RNA-guided endonuclease.

21. The method of any one of claims 1-20, wherein the RNA-guided endonuclease is a Cas9 endonuclease.

22. The method of claim 21, wherein the Cas9 endonuclease is S. pyogenes Cas9, S. aureus Cas9, N. meningitides Cas9, S. thermophilus Cas9, S. thermophilus 3 Cas9, T. denticola Cas9, or a variant thereof.

23. The method of any one of claims 1-22, wherein the gRNA is a single-guide RNA (sgRNA).

24. The method of any one of claims 1-23, wherein the gRNA targets exon 3 of LPA gene.

25. The method of any one of claims 1-24, wherein the gRNA comprises a spacer sequence of any one of SEQ ID NOs: 18-25.

26. The method of any one of claims 1-24, wherein the LPA gRNA comprises the spacer sequence of SEQ ID NO: 18.

27. The method of any one of claims 1-24, wherein the LPA gRNA is a single guide RNA (sgRNA) comprising the sequence of SEQ ID NO: 32.

28. The method of any one of claims 1-24, wherein the LPA gRNA is a single guide RNA (sgRNA) comprising the sequence of SEQ ID NO: 11.

29. The method of any one of claims 1-28, wherein the LPA gRNA or the nucleic acid encoding the LPA gRNA, and the RNA-guided nuclease are encapsulated in the plurality of nanoparticles.

30. The method of claim 29, wherein the nanoparticles are lipid nanoparticles.

31. The method of any one of claims 1-30, wherein the subject is a primate subject.

32. The method of any one of claims 1-31, wherein the subject is human.

33. The method of any one of claims 1-32, wherein the method comprises a single administration of the plurality of nanoparticles to the subject.

34. The method of claim 33, wherein the plurality of nanoparticles is administered to the subject in the single administration at a dose of, or a dose of about, 0.1 mg/kg, 0.3 mg/kg, 0.6 mg/kg, or 1 mg/kg of total nucleic acids of (a) and (b).

35. The method of any one of claims 1-34, wherein the plurality of nanoparticles are lipid nanoparticles.

36. The method of claim 35, wherein the lipid nanoparticles comprise one or more neutral lipids, charged lipids, ionizable lipids, steroids, and polymers conjugated lipids.

37. The method of claim 35, wherein the lipid nanoparticles comprise cholesterol, a polyethylene glycol (PEG) lipid, or both.

38. The method of any one of claims 1-37, further comprising determining (i) a level of one or more of alanine transaminase (ALT), aspartate transaminase (AST), gamma-glutamyl transferase (GGT), bilirubin, alkaline phosphatase (Alk Phos) and albumin; (ii) prothrombin time (PT), and/or (iii) partial thromboplastin time (PTT) in the subject before the administration, after the administration, or both.

39. The method of any one of claims 1-38, wherein the subject is administered an additional treatment, wherein the additional treatment comprises administration of a corticosteroid, an anti-H1 antihistamine, an anti-H2 antihistamine, or any combination thereof.

40. The method of claim 39, wherein the additional treatment is administered to the subject 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, or more prior to the administration of the plurality of nanoparticles to the subject.

41. The method of any one of claims 39-40, wherein the additional treatment and the plurality of nanoparticles are administered simultaneously.

42. A composition, comprising a plurality of nanoparticles complexed with (a) a guide RNA (gRNA) targeting LPA gene (LPA gRNA), and (b) an mRNA encoding Cas9 endonuclease, wherein the gRNA comprises a spacer sequence of any one of SEQ ID NOs: 18-25.

43. A composition for use in the treatment of a lipoprotein-related diseases, comprising a plurality of nanoparticles complexed with (a) a guide RNA (gRNA) targeting LPA gene (LPA gRNA), and (b) an mRNA encoding Cas9 endonuclease, wherein the gRNA comprises a spacer sequence of any one of SEQ ID NOs: 18-25.

44. The composition of claim 43, wherein the lipoprotein-related disease is a metabolic disease, a cardiovascular disease, a lipid metabolism disease, or a combination thereof.

45. The composition of claim 43, wherein the lipoprotein-related disease is calcific aortic valve disease, myocardial infarctions, coronary heart disease, atherosclerosis, thrombosis, stroke, coronary artery disease, familial hyperlipidemia, myocardial infarction, peripheral arterial disease, calcific aortic valve stenosis, or a combination thereof.

46. The composition of any one of claims 42-45, wherein the gRNA comprises the spacer sequence of SEQ ID NO: 18.

47. The composition of any one of claims 42-45, wherein the gRNA comprises the sequence of SEQ ID NO: 32.

48. The composition of any one of claims 42-45, wherein the gRNA comprises the sequence of SEQ ID NO: 11.

49. The composition of any one of claims 42-48, wherein the Cas9 endonuclease is S. pyogenes Cas9 endonuclease.

50. The composition of any one of claims 42-49, wherein the plurality of nanoparticles are lipid nanoparticles.

51. The composition of claim 50, wherein the lipid nanoparticles comprise one or more neutral lipids, charged lipids, ionizable lipids, steroids, and polymers conjugated lipids.

52. The composition of claim 50, wherein the lipid nanoparticles comprise cholesterol, a polyethylene glycol (PEG) lipid, or both.

53. The composition of any one of claims 42-52, wherein the composition is a pharmaceutical composition comprising one or more pharmaceutically acceptable excipients.
